(19) United States
(12) Patent Application Publication
(10) Pub. No.: US 2007/0249618 A1
(43) Pub. Date: Oct. 25, 2007

(54) Novel Piperidine Derivatives as Histamine H3 Receptor Ligands for Treatment of Depression

(75) Inventors: James Folmer, Wilmington, DE (US); Simon Fraser Hunt, Leicestershire (GB); Peter Hamley, Koenigstein (DE); Steven Wesolowski, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/572,966

(22) PCT Filed: Jul. 27, 2005
(86) PCT No.: PCT/SE05/01188

§ 371 Date: Jan. 30, 2007

(30) Foreign Application Priority Data
Aug. 2, 2004 (SE) 0401970-9

(51) Int. Cl. A61K 31/496; A61K 31/4523; A61K 31/4545; C07D 295/14; C07D 401/14; C07D 405/12; C07D 413/12; C07D 471/10; C07D 417/12; C07D 409/14; C07D 403/12; C07D 401/06; C07D 241/04; A61K 31/495

(52) U.S. Cl. 514/252.11; 514/253.01; 514/253.1; 514/255.01; 514/316; 514/317; 544/121; 544/357; 544/363; 544/365; 544/367; 544/368; 544/372; 544/373; 544/377; 544/379; 544/390; 546/20

(57) ABSTRACT

Compounds of formula (I) wherein Ar¹ and Q are as defined in the specification, as well as salts, enantiomers thereof and pharmaceutical compositions including the compounds are prepared. They are useful in therapy, in particular in the treatment of depression.

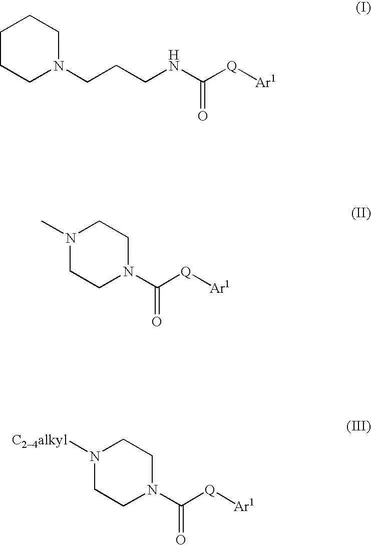

### FIELD OF THE INVENTION

This invention relates histamine receptor ligands. More specifically, the invention relates to histamine H3 receptor ligands, preparation thereof and uses thereof.

### BACKGROUND OF THE INVENTION

The histamine H3 receptor is of current interest for the development of new medicaments. This receptor is a presynaptic autoreceptor located both in the central and the peripheral nervous system, the skin and in organs such as the lung, the intestine, probably the spleen and the gastrointestinal tract. Recent evidence suggests that the H3 receptor shows intrinsic, constitutive activity, in vitro as well as in vivo (i.e., it is active in the absence of an agonist. Compounds acting as inverse agonists can inhibit this activity. The histamine H3 receptor has been demonstrated to regulate the release of histamine and also of other neurotransmitters such as serotonin and acetylcholine. Some histamine H3 ligands such as histamine H3 receptor antagonists or inverse agonists may increase the release of these neurotransmitters in the brain whereas other histamine H3 ligands such as histamine H3 receptor agonists may lead to an inhibition of the biosynthesis of histamine and an inhibition of the release of histamine and also of other neurotransmitters. This suggests that histamine H3 receptor agonists, inverse agonists and antagonists could be mediators of neuronal activity. Accordingly, the histamine H3 receptor may be a target for new therapeutics.

There are publications that disclose the preparation and use of imidazole derivative histamine H3 ligands. However, there are needs for additional histamine H3 ligands.

### DESCRIPTION OF THE INVENTION

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4 n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4 n+2 delocalized electrons) and comprising 5 up to about 14 carbon atoms, which serves to link two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4 n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₆hydrocarbon groups, or one or more chemical groups containing one or more heteroatoms selected from N, O, S, F, Cl, Br, I, and P. Exemplary chemical groups containing one or more heteroatoms include—NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₆hydrocarbyl. For example, substituted phenyl may refer to nitrophenyl, methoxyphenyl, chlorophenyl, aminophenyl, etc., wherein the nitro, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzoflranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo [2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to radicals of the general formula —NRR′, wherein R and R′ are independently selected from hydrogen or a hydrocarbon radical.

Halogen includes fluorine, chlorine, bromine and iodine.

“Halogenated,” used as a prefix of a group, means one or more hydrogens on the group is replaced with one or more halogens.

“RT” or “rt” means room temperature.

In one aspect, the invention provides a compound of formula I, II or III, a pharmaceutically acceptable salt thereof, diastereomers thereof, enantiomers thereof, and mixtures thereof: 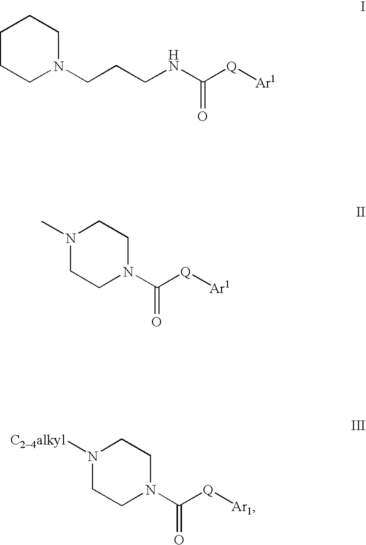
wherein

Ar¹ is selected from C₆₋₁₀aryl and C₂₋₉heteroaryl, wherein said C₆₋₁₀aryl and C₂₋₉heteroaryl are optionally substituted with one or more groups selected from —R, —NO₂, —OR, —Cl, —Br, —I, —F, —CF₃, —OCF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₃₋₆cycloalkyl, C₃₋₆heterocyclyl, phenyl, benzyl, C₁₋₆alkyl or C₂₋₆alkenyl and wherein said R is further optionally substituted with one or more groups selected from methyl, methoxy, hydroxy and halogen; and

Q is a divalent or trivalent group that connects the carbonyl with Ar¹, wherein said divalent or trivalent group contains at least one nitrogen, said nitrogen is directly connected to the carbonyl group of formula I, II or III to form an amide bond therebetween, and said trivalent group is fused with Ar¹.

In one embodiment, the compound of the present invention may be a compound of formula I, wherein Ar¹ is represented by

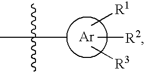

wherein Ar is selected from phenyl, pyridyl, naphthyl, 1,2,3,4-tetrahydro-naphthyl; thienyl, furyl, thiazolyl, benzo[1,3]dioxolyl, 4,5,6,7-tetrahydro-thieno[2,3-c]pyridinyl; 2,3-dihydro-benzo[1,4]dioxinyl; quinolyl; isoquinolyl; indolyl; pyrroyl, benzotriazolyl; benzoimidazolyl, 2,3-dihydro-benzoffiranyl; 2,3-dihydro-isoindol-1-on-yl; benzo[1,2,3]thiadiazolyl, benzothiazolyl, and 4H-benzo[1,4]oxazin-3-on-yl;

R¹, R² and R³ are independently selected from —R, —NO₂, —OR, —Cl, —Br, —I, —F, —CF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₅₋₆cycloalkyl, C₃₋₅heterocyclyl, phenyl, benzyl, C₁₋₄alkyl or C₂₋₄alkenyl, and wherein said R is further optionally substituted with one or more groups selected from methyl, methoxy, hydroxy and halogen;

Q is selected from: 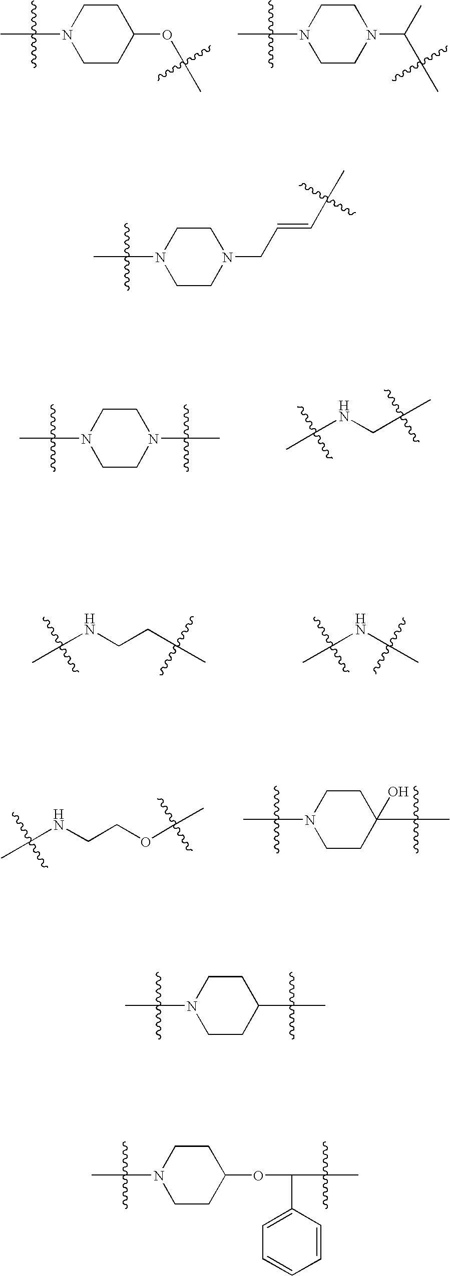 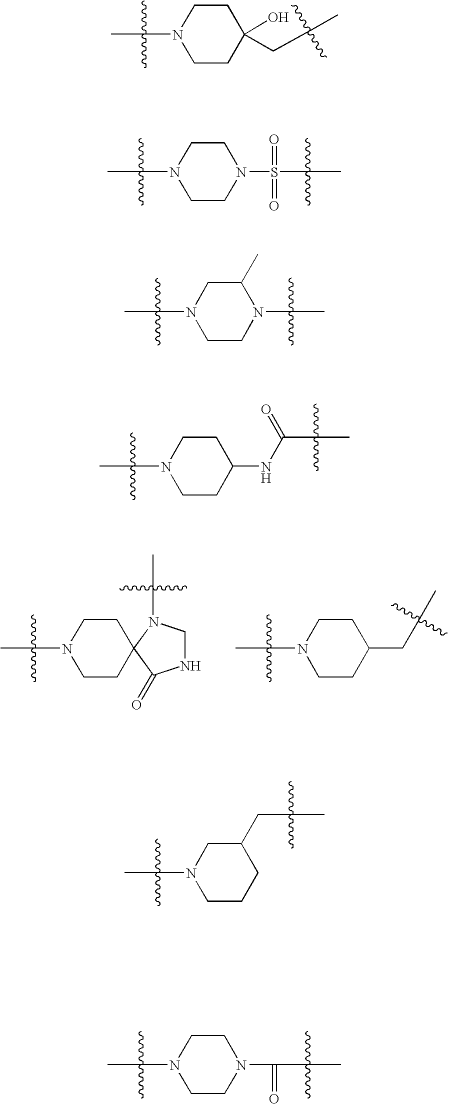
or Q may be a trivalent group such as 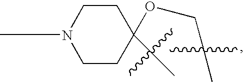
which is fused with Ar¹, wherein Ar¹ is a divalent aromatic group such as 1,2-phenylene.

In another embodiment, the compounds of the present invention are represented by formula I, wherein Ar¹ is selected from phenyl, 2-pyridyl, 4-pyridyl; 1-naphthyl, 2-naphthyl, 1,2,3,4-tetrahydro-naphth-1-yl; 1,2,3,4-tetrahydro-naphth-5-yl; 2-thienyl, 3-thienyl, 2-furyl, 2-thiazolyl, benzo[1,3]dioxol-5-yl, 4,5,6,7-tetrahydro-thieno[2,3-c]pyridin-2-yl; 2,3-dihydro-benzo[1,4]dioxin-6-yl; 2,3-dihydro-benzo[1,4]dioxin-2-yl; quinol-2-yl, isoquinol-5-yl; 1H-indol-4-yl, 1H-indol-3-yl, 1H-indol-2-yl, 1H-indol-7-yl, 1-pyrroyl, 1H-benzotriazol-5-yl, 1H-benzoimidazol-5-yl, 2,3-dihydro-benzofuran-5-yl, 2,3-dihydro-isoindol-1-on-2-yl; benzo[1,2,3]thiadiazol-5-yl, benzo[1,2,3]thiadiazol-6-yl, benzothiazol-6-yl, benzothiazol-2-yl, and 4H-benzo[1,4]oxazin-3-on-7-yl, wherein Ar¹ is further optionally substituted with one or more groups selected from C₁₋₄alkyl, C₂₋₄alkenyl, C₁₋₄alkoxy, C₁₋₄alkenyloxy, phenoxy, 4-methoxyphenoxy, benzyl, acetoamino, methylsulfonyl, methoxycarbonyl, nitro, chloro, fluoro, bromo, iodo, 1-pyrroyl, 2-methyl-pyrro-1-yl, amino, phenylsulfonyl, aceto, 1-piperidinyl, [1,2,3]thiadiazol-4-yl, 4-morpholinyl, methoxy, ethoxy, isopropyloxy, methythio, cyano, dimethylamino, hydroxy, methylaminosulfonyl, trifluoromethyl, trifluoromethoxy, phenyl, phenoxy, benzyl, 4-hydroxyl-phenyl, diethylamino, methylsulfonyl, aminosulfonyl, cyclohexyl, 1-pyrrolyl, 1H-pyrazol-3-yl, 5-tetrazolyl, 1-piperidinyl, 1-pyrazolyl, methylsulfonylmethyl, 3,5-dimethyl-pyrazolyl, pyrrolidin-2-on-1-yl; and

Q is selected from

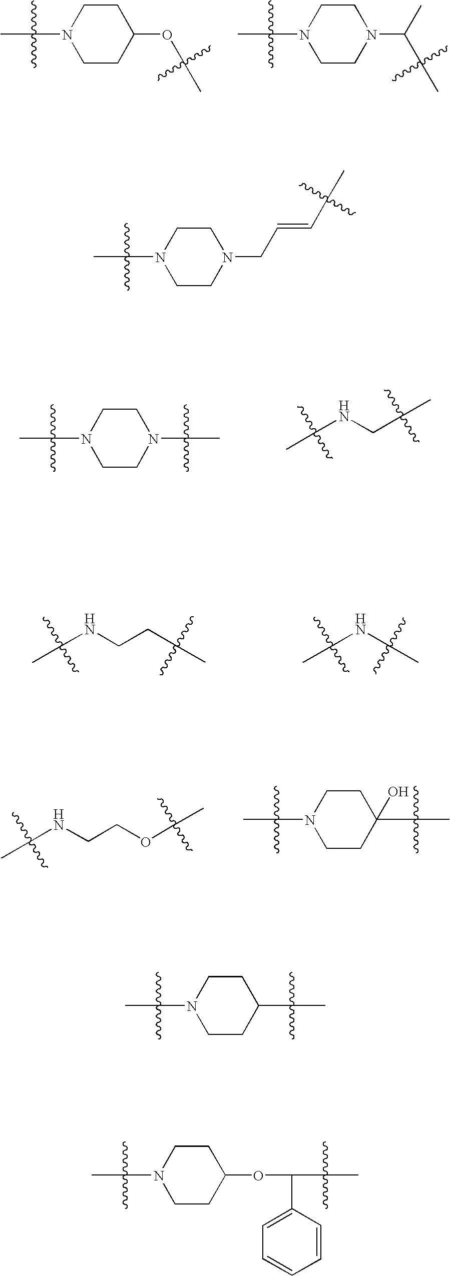

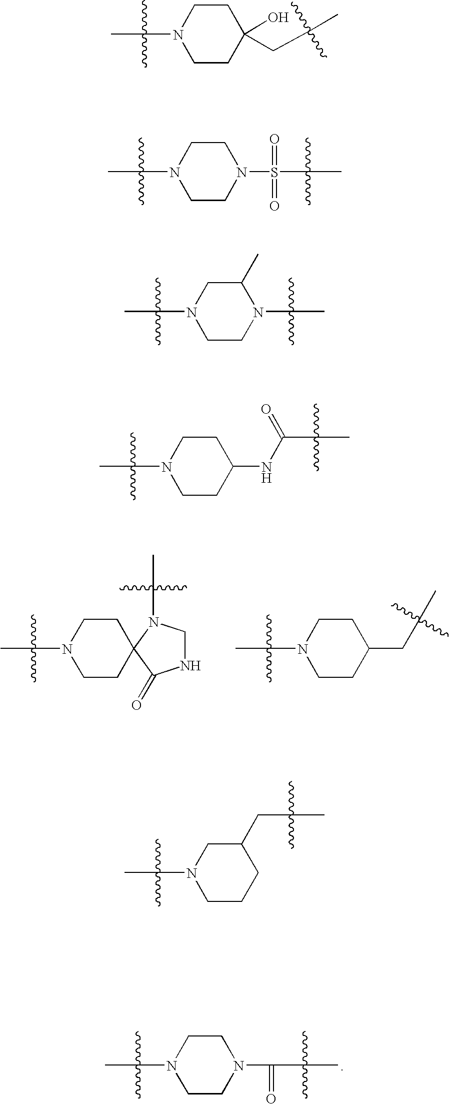

In a further embodiment, the compounds of the present invention are selected from 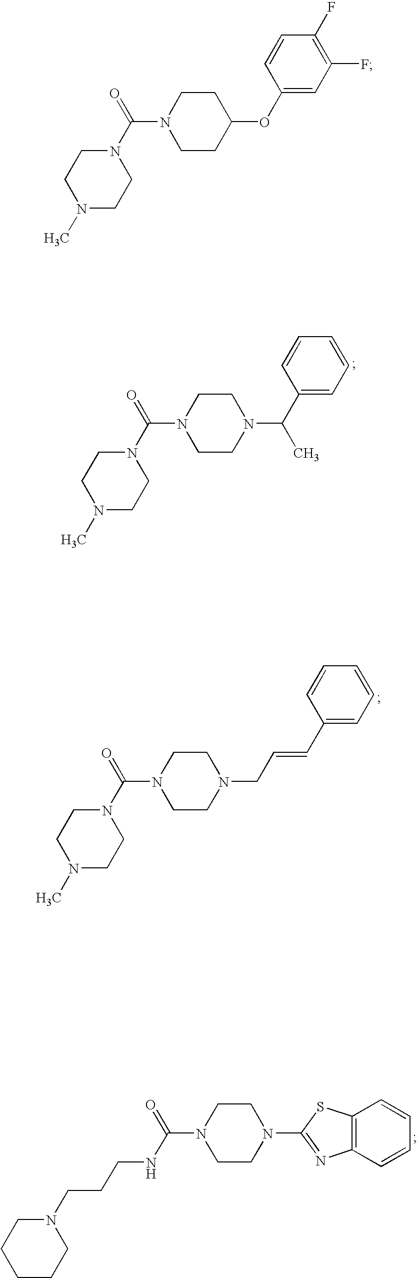 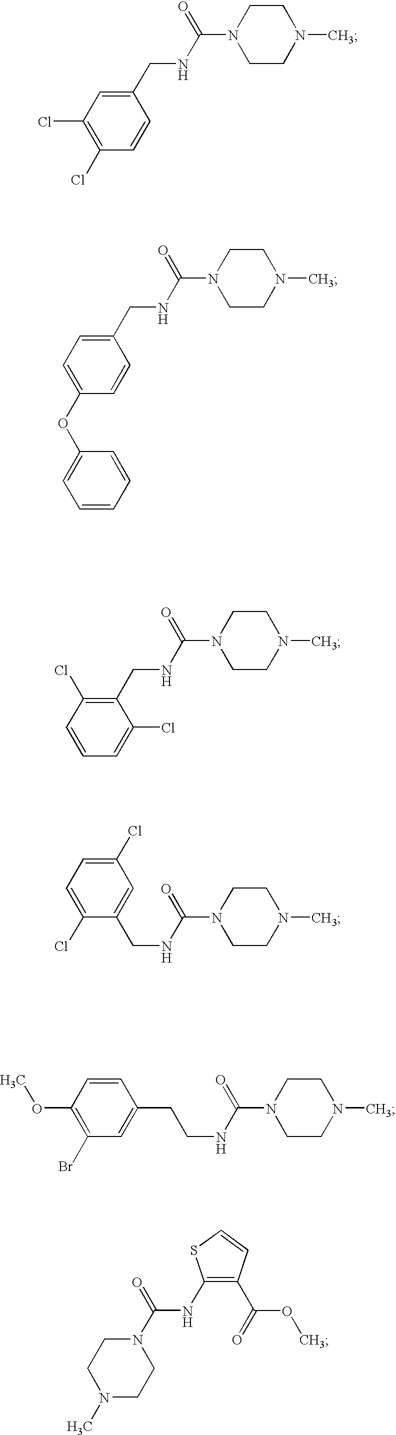 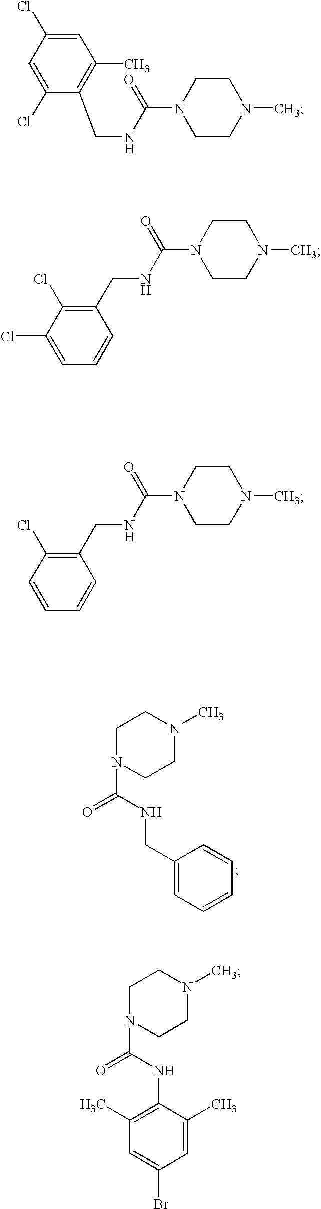 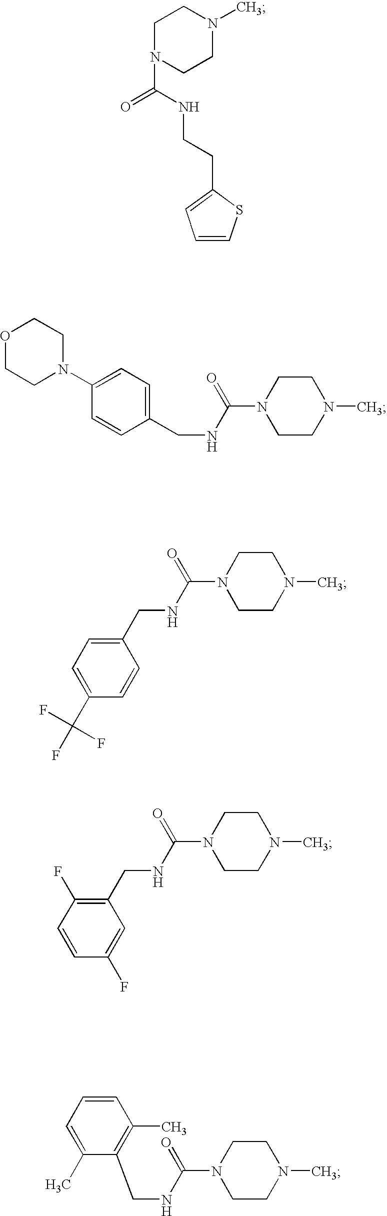 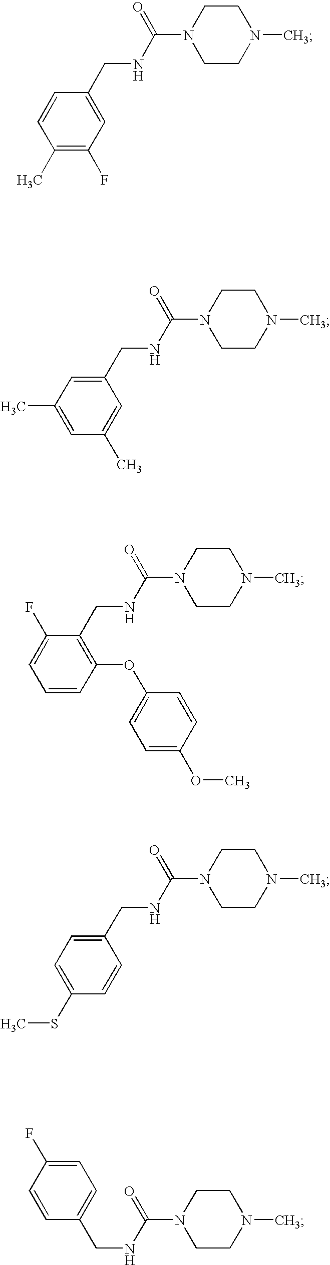 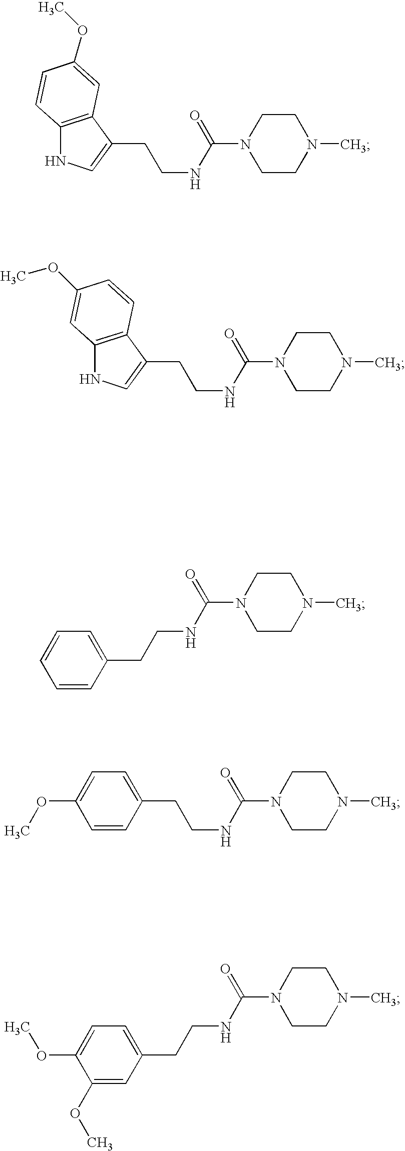 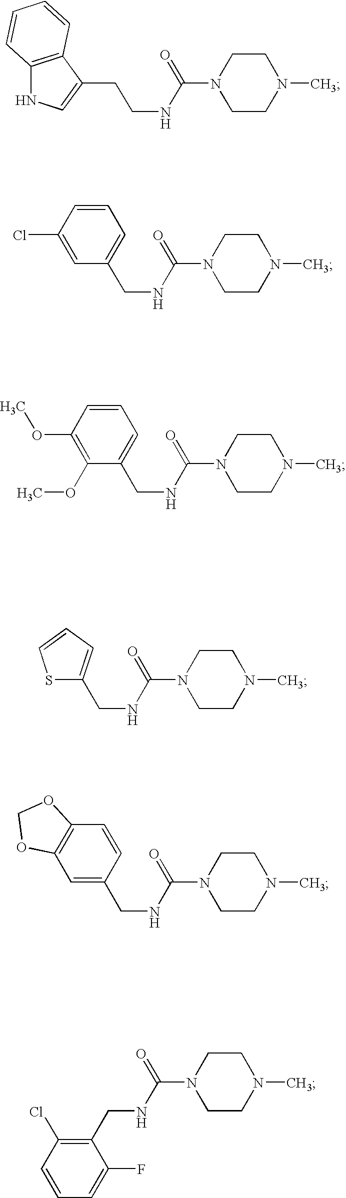 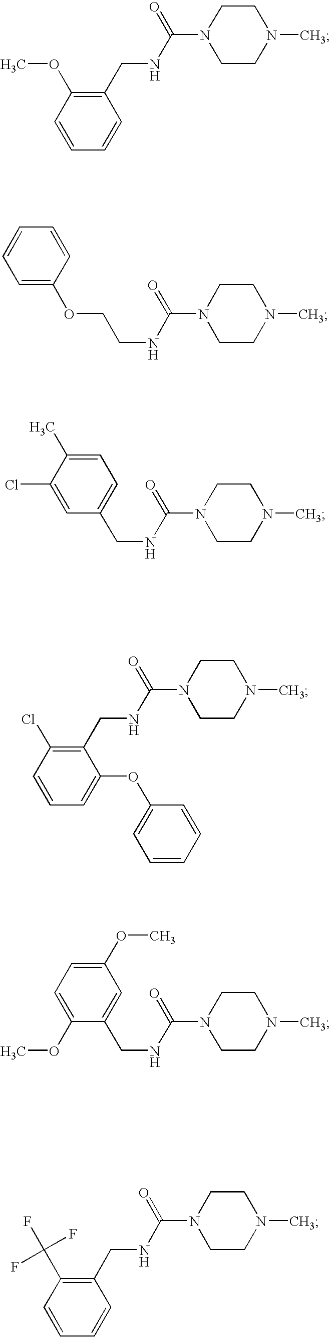 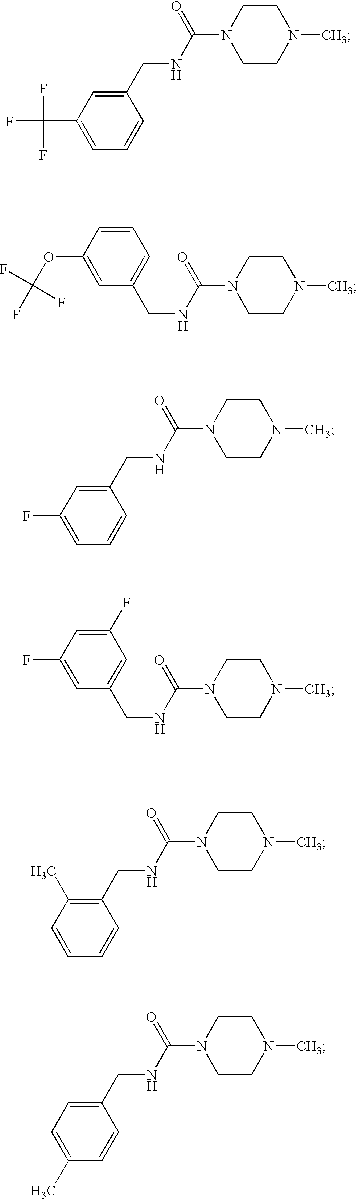 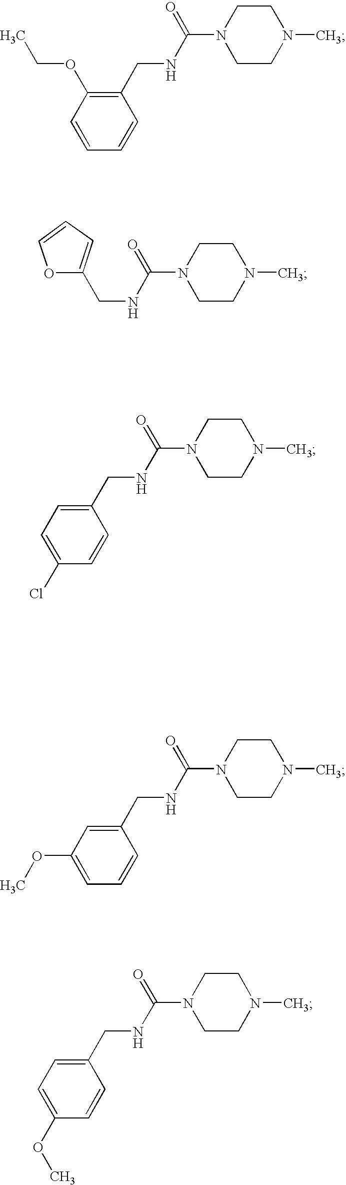 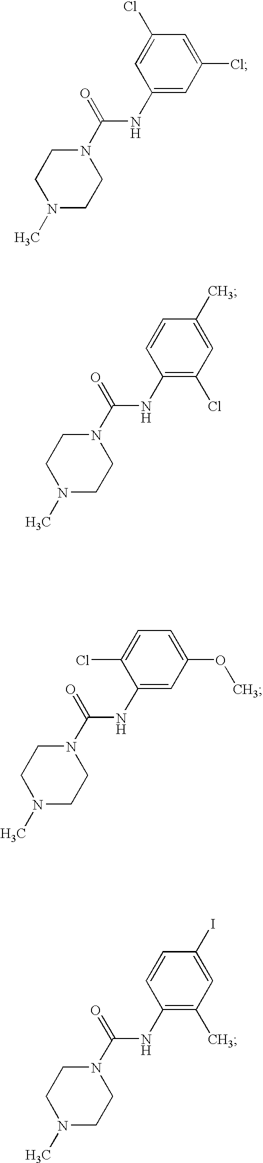 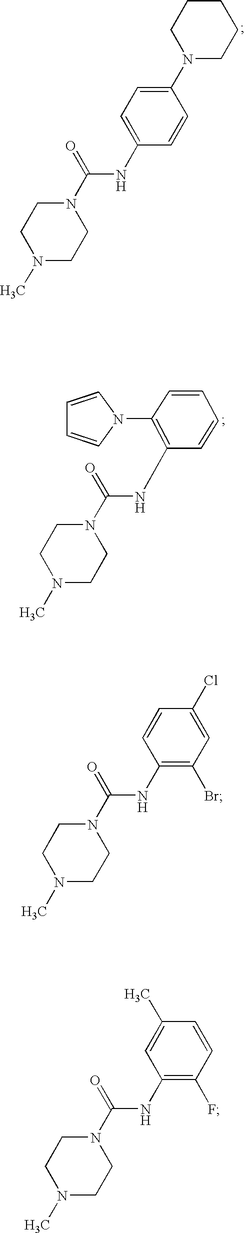 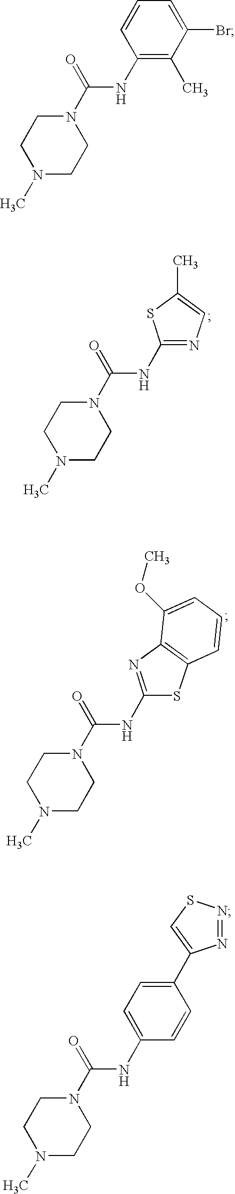 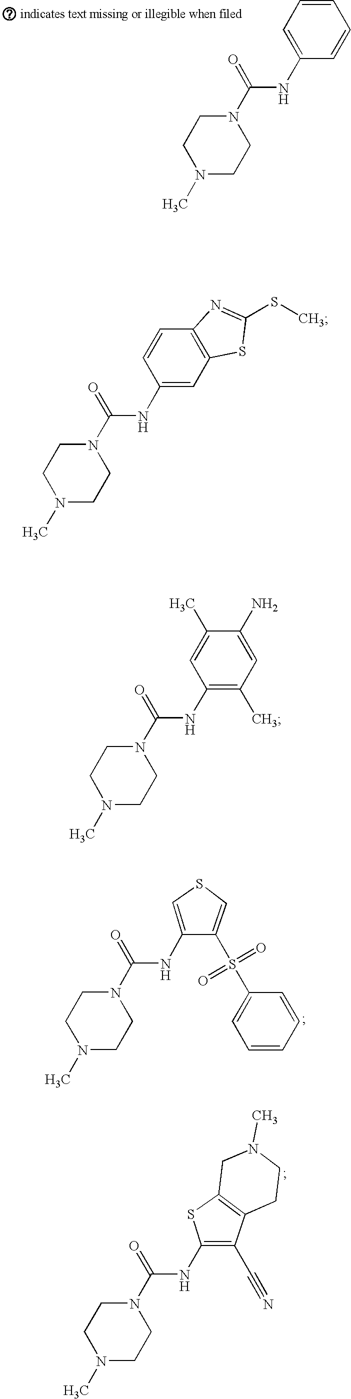 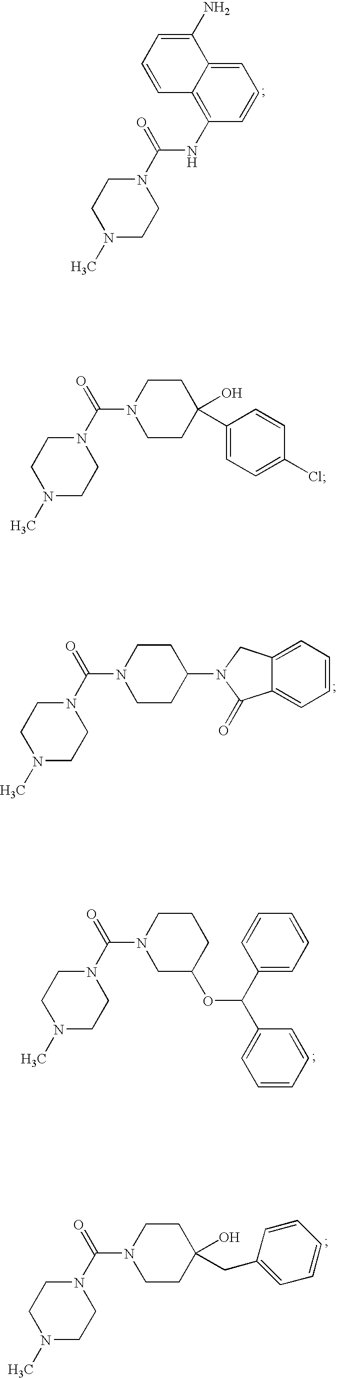 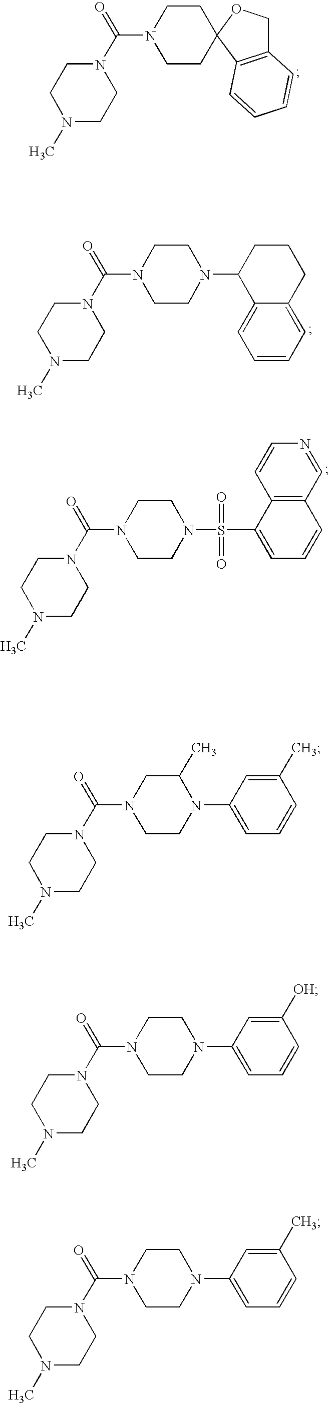 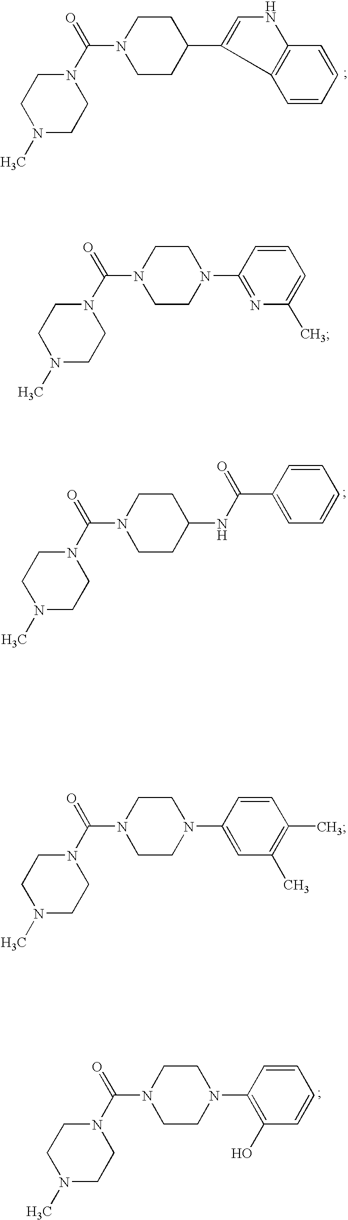 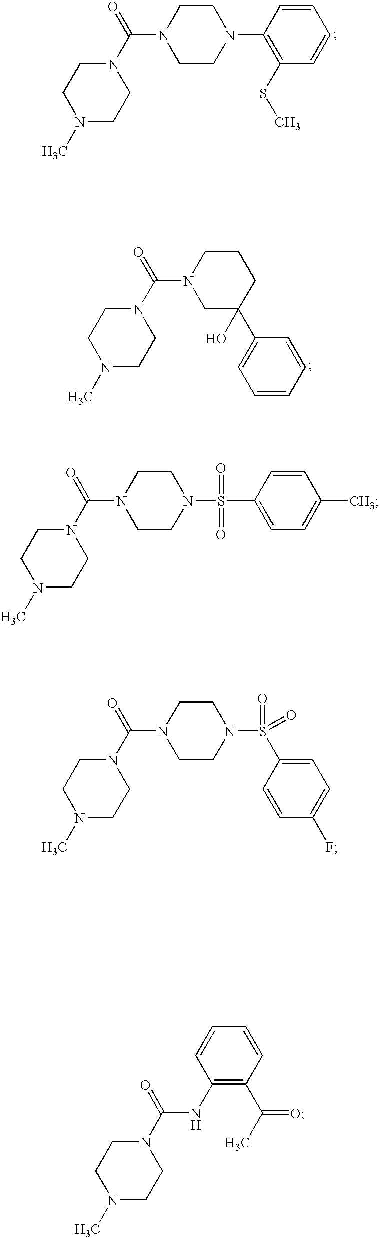 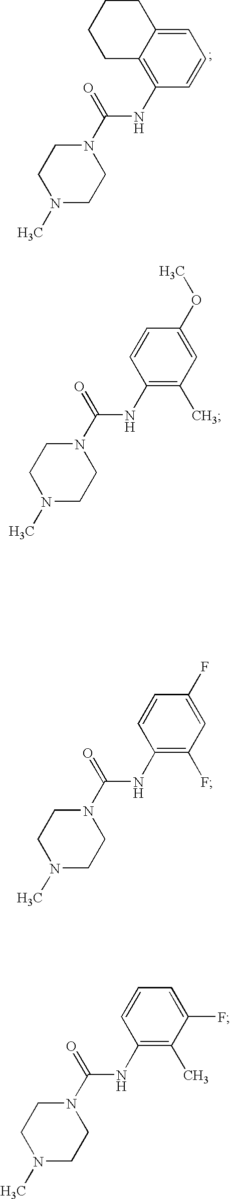 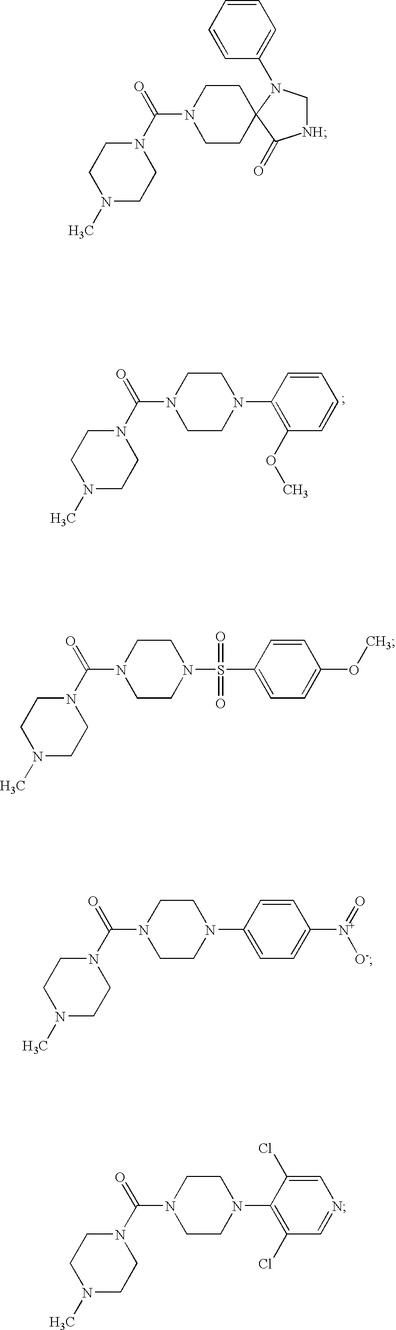 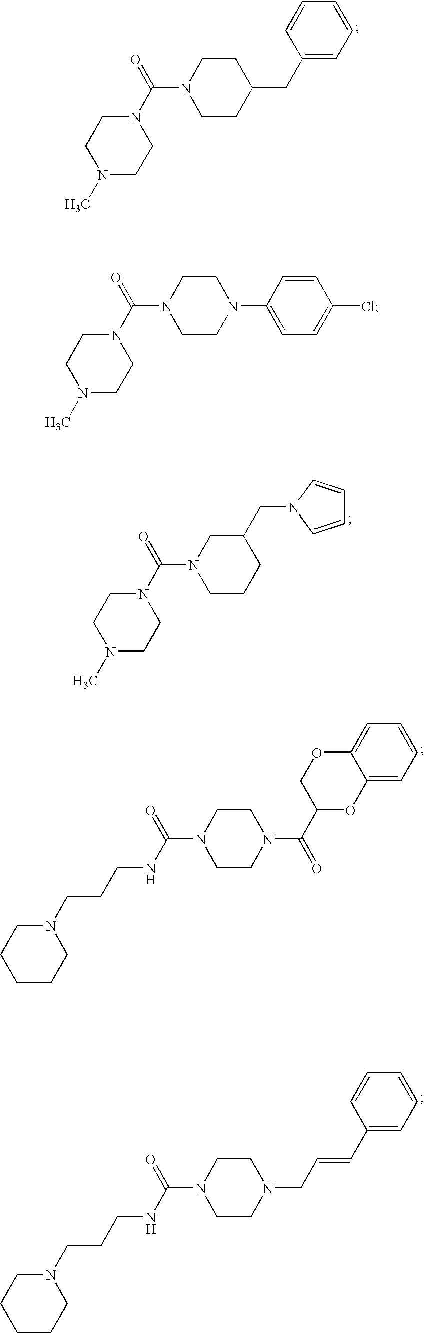 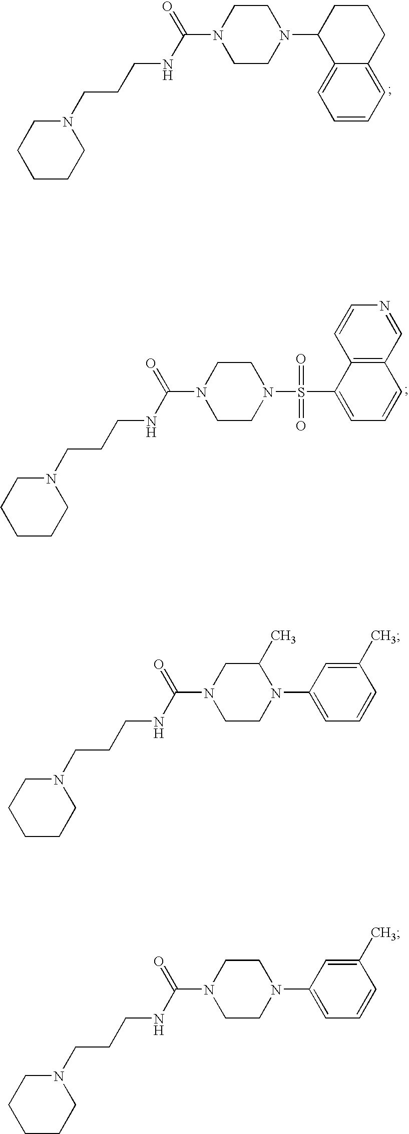 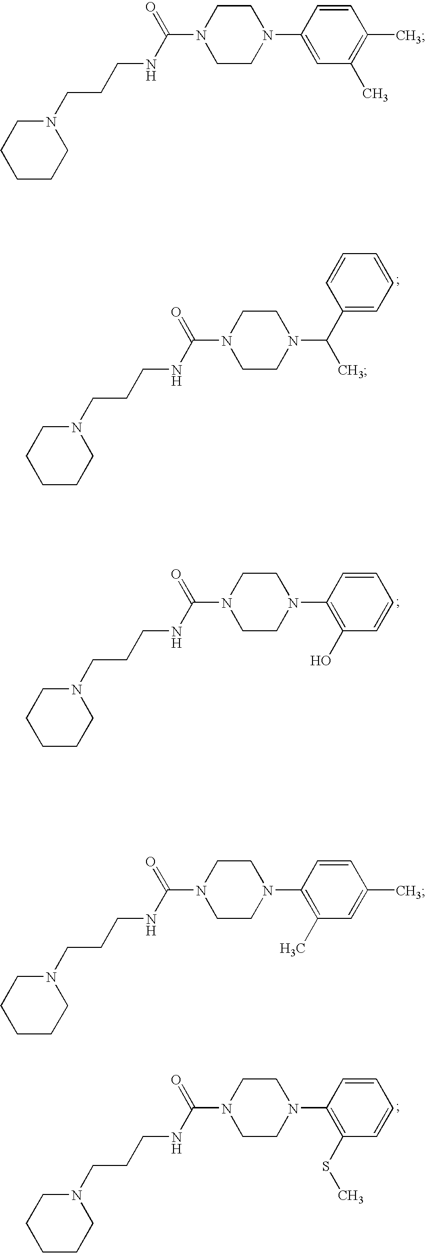 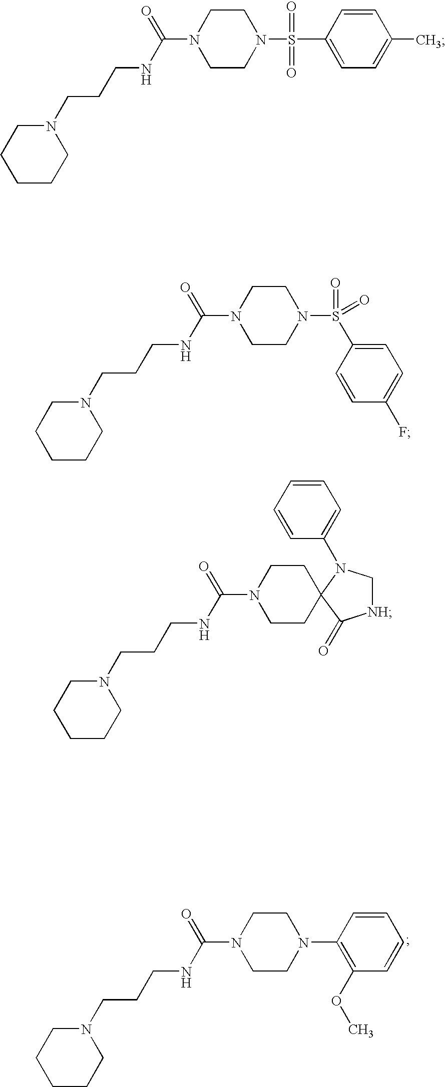 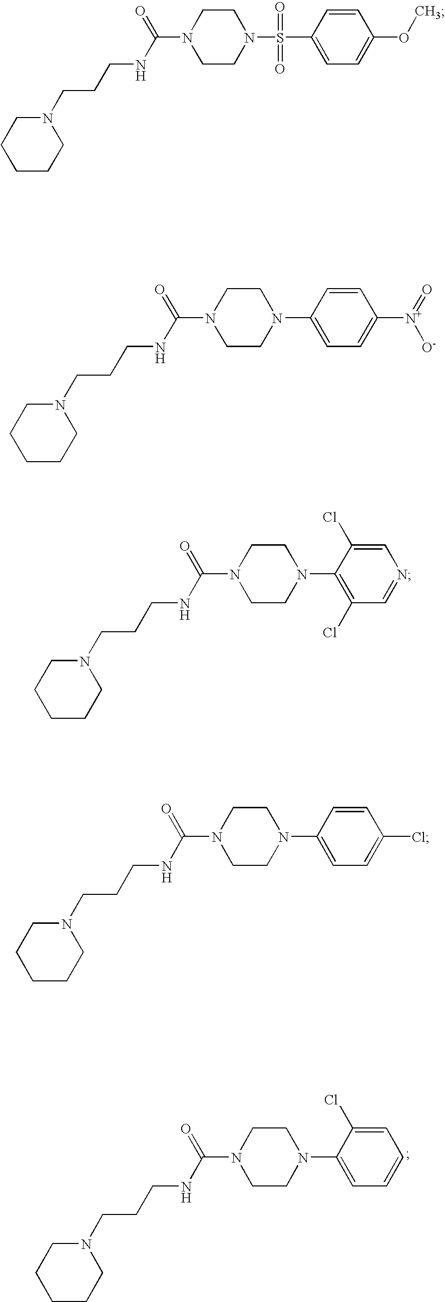
pharmaceutically acceptable salts thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I or II. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I or II. It will further be understood that the present invention encompasses tautomers of the compounds of the formula I, II or III.

It will also be understood that certain compounds of the present invention may exist in solvated, for example hydrated, as well as unsolvated forms. It will farther be understood that the present invention encompasses all such solvated forms of the compounds of the formula I, II or III.

Within the scope of the invention are also salts of the compounds of the formula I, II or III. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I, II or III above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The compounds of the present invention are useful in the treatment of a wide range of conditions and disorders in which an interaction with the histamine H3 receptor is beneficial. Thus, the compounds may find use e.g. in the treatment of diseases of the central nervous system, the peripheral nervous system, the cardiovascular system, the pulmonary system, the gastrointestinal system and the endocrinological system.

The compounds of the present invention are useful in therapy, especially for the treatment of various depression conditions.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful for the treatment of obesity, epilepsy, Alzheimer's disease, dementia, schizophrenia, cognitive defect, rhinitis, cognition disorders, central nervous system disease, neurological disorder, epilepsy, attention deficit hyperactivity disorder, eating disorder, allergic rhinitis, allergy, inflammation, migraine, sleep disorder, narcolepsy, anxiety disorder, psychiatric conditions, depression, multiple sclerosis, anxiety, bipolar disorder, stroke, sleep disorder, mental disorder, cognitive disorder and non-insulin dependent diabetes.

Compounds of the invention are useful as an anti-depression agent. Combinations of agents with different properties may be used to achieve a balance of effects needed to treat depression.

Also within the scope of the invention is the use of any of the compounds according to the formula I, II or III above, for the manufacture of a medicament for the treatment of any of the conditions discussed above.

A further aspect of the invention is a method for the treatment of a subject suffering from any of the conditions discussed above, whereby an effective amount of a compound according to the formula I, II or III above, is administered to a patient in need of such treatment.

Thus, the invention provides a compound of formula I, II or III, or pharmaceutically acceptable salt or solvate thereof, as hereinbefore defined for use in therapy.

In a further aspect, the present invention provides the use of a compound of formula I, II or III, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (per cent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I, II or III as defined above for the manufacture of a medicament.

Also within the scope of the invention is the use of any compound of formula I, II or III for the manufacture of a medicament for the therapy of depression.

Additionally provided is the use of any compound according to Formula I or II for the manufacture of a medicament for the therapy of various depression conditions.

A further aspect of the invention is a method for therapy of a subject suffering from any of the conditions discussed above, whereby an effective amount of a compound according to the formula I, II or III above, is administered to a patient in need of such therapy.

Additionally, there is provided a pharmaceutical composition comprising a compound of Formula I or II, or a pharmaceutically acceptable salt thereof, in association with a pharmaceutically acceptable carrier.

Particularly, there is provided a pharmaceutical composition comprising a compound of Formula I or II, or a pharmaceutically acceptable salt thereof, in association with a pharmaceutically acceptable carrier for therapy, more particularly for therapy of depression.

Further, there is provided a pharmaceutical composition comprising a compound of Formula I or II, or a pharmaceutically acceptable salt thereof, in association with a pharmaceutically acceptable carrier use in any of the conditions discussed above.

In a further aspect, the invention provides a process for preparing a compound of formula I, comprising:

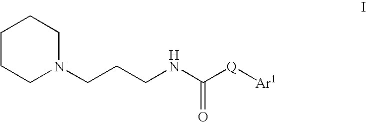

reacting Ar¹-Q-H with 3-(1-piperidino)propylamine and a haloformate, Ar¹ is selected from C₆₋₁₀aryl and C₂₋₉heteroaryl, wherein said C₆₋₁₀aryl and C₂₋₉heteroaryl are optionally substituted with one or more groups selected from —R, —NO₂, —OR, —Cl, —Br, —I, —F, —CF₃, —OCF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₃₋₆cycloalkyl, C₃₋₆heterocyclyl, phenyl, benzyl, C₁₋₆alkyl or C₂₋₆alkenyl and wherein said R is further optionally substituted with one or more groups selected from methyl, methoxy, hydroxy and halogen;

Q is a divalent or trivalent group that connects the carbonyl with Ar¹, wherein said divalent or trivalent group contains at least one nitrogen, wherein said nitrogen of Q is connected to the H in Ar¹-Q-H to form an amino, and said trivalent group is fused with Ar¹; and said Q-H of Ar¹-Q-H forms an amino group.

In one embodiment, the process of preparing a compound of formula I comprising:

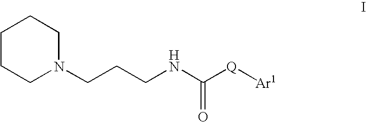

combining Ar¹-Q-H with 3-(1-piperidino)propylamine and a haloformate,

wherein Ar¹ is selected from phenyl, 2-pyridyl, 4-pyridyl; 1-naphthyl, 2-naphthyl, 1,2,3,4-tetrahydro-naphth-1-yl; 1,2,3,4-tetrahydro-naphth-5-yl; 2-thienyl, 3-thienyl, 2-furyl, 2-thiazolyl, benzo[1,3]dioxol-5-yl, 4,5,6,7-tetrahydro-thieno[2,3-c]pyridin-2-yl; 2,3-dihydro-benzo[1,4]dioxin-6-yl; 2,3-dihydro-benzo[1,4]dioxin-2-yl; quinol-2-yl, isoquinol-5-yl; 1H-indol-4-yl, 1H-indol-3-yl, 1H-indol-2-yl, 1H-indol-7-yl, 1-pyrroyl, 1H-benzotriazol-5-yl, 1H-benzoimidazol-5-yl, 2,3-dihydro-benzofuran-5-yl, 2,3-dihydro-isoindol-1-on-2-yl; benzo[1,2,3]thiadiazol-5-yl, benzo[1,2,3]thiadiazol-6-yl, benzothiazol-6-yl, benzothiazol-2-yl, and 4H-benzo[1,4]oxazin-3-on-7-yl, wherein Ar¹ is further optionally substituted with one or more groups selected from C₁₋₄alkyl, C₂₋₄alkenyl, C₁₋₄alkoxy, C₁₋₄alkenyloxy, phenoxy, 4-methoxyphenoxy, benzyl, acetoamino, methylsulfonyl, methoxycarbonyl, nitro, chloro, fluoro, bromo, iodo, 1-pyrroyl, 2-methyl-pyrro-1-yl, amino, phenylsulfonyl, aceto,1-piperidinyl, [1,2,3]thiadiazol-4-yl, 4-morpholinyl, methoxy, ethoxy, isopropyloxy, methythio, cyano, dimethylamino, hydroxy, methylaminosulfonyl, trifluoromethyl, trifluoromethoxy, phenyl, phenoxy, benzyl, 4-hydroxyl-phenyl, diethylamino, methylsulfonyl, aminosulfonyl, cyclohexyl, 1-pyrrolyl, 1H-pyrazol-3-yl, 5-tetrazolyl, 1-piperidinyl, 1-pyrazolyl, methylsulfonylmethyl, 3,5-dimethyl-pyrazolyl, pyrrolidin-2-on-1-yl; and

Q is selected from 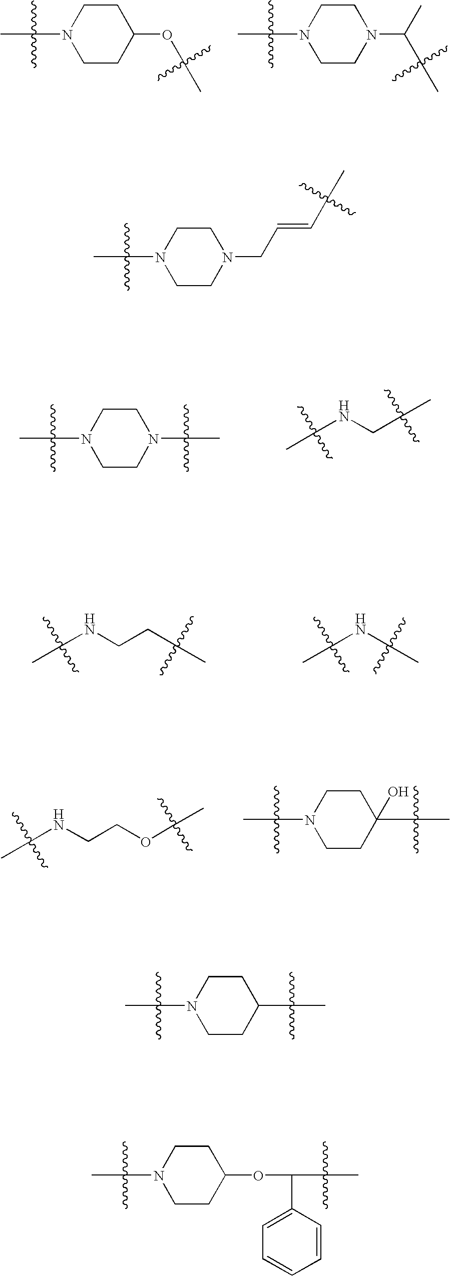 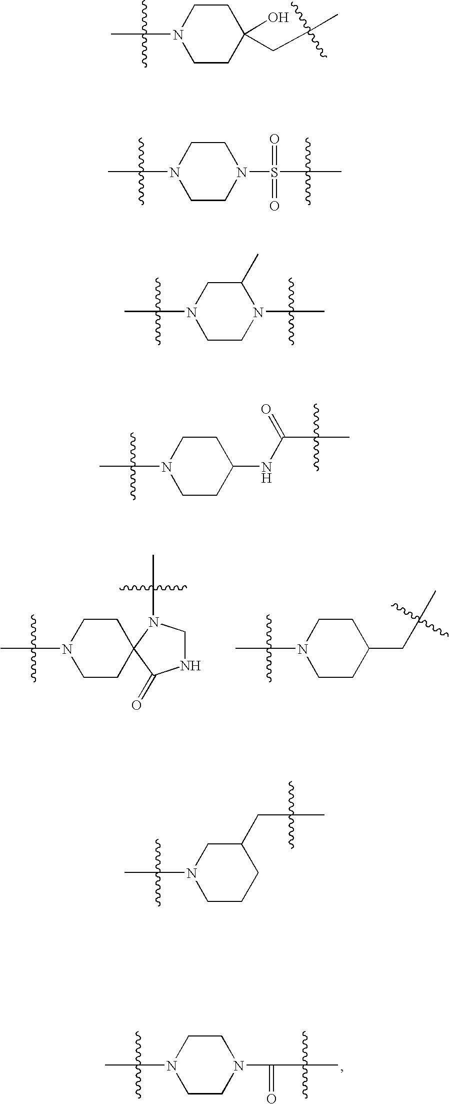
and wherein the left side nitrogen atom in the above-identified structures of Q is connected to the H in Ar¹-Q-H to form an amino group

In a particular embodiment, the step of combining Ar¹-Q-H with 3-(1-piperidino)propylamine and a haloformate may be carried out at ambient temperature and in the presence of organic base such as diisopropylethylamine. The haloformate may be 4-nitrophenyl chloroformate.

In a further aspect, the invention provides a process for preparing a compound of formula II, comprising:

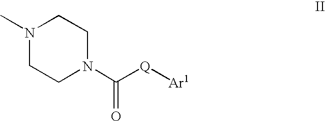

reacting Ar¹-Q-H with N-methyl piperazine and a haloformate,

Ar¹ is selected from C₆₋₁₀aryl and C₂₋₉heteroaryl, wherein said C₆₋₁₀aryl and C₂₋₉heteroaryl are optionally substituted with one or more groups selected from —R, —NO₂, —OR, —Cl, —Br, —I, —F, —CF₃, —OCF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₃₋₆cycloalkyl, C₃₋₆heterocyclyl, phenyl, benzyl, C₁₋₆alkyl or C₂₋₆alkenyl and wherein said R is further optionally substituted with one or more groups selected from methyl, methoxy, hydroxy and halogen;

Q is a divalent or trivalent group that connects the carbonyl with Ar¹, wherein said divalent or trivalent group contains at least one nitrogen, wherein said nitrogen of Q is connected to the H in Ar¹-Q-H to form an amino, and said trivalent group is fused with Ar¹; and said Q-H of Ar¹-Q-H forms an amino group.

In one embodiment, the process of preparing a compound of formula II comprising:

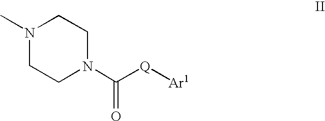

combining Ar¹-Q-H with N-methyl piperazine and a haloformate,

wherein Ar¹ is selected from phenyl, 2-pyridyl, 4-pyridyl; 1-naphthyl, 2-naphthyl, 1,2,3,4-tetrahydro-naphth-1-yl; 1,2,3,4-tetrahydro-naphth-5-yl; 2-thienyl, 3-thienyl, 2-furyl, 2-thiazolyl, benzo[1,3]dioxol-5-yl, 4,5,6,7-tetrahydro-thieno[2,3-c]pyridin-2-yl; 2,3-dihydro-benzo[1,4]dioxin-6-yl; 2,3-dihydro-benzo[1,4]dioxin-2-yl; quinol-2-yl, isoquinol-5-yl; 1H-indol-4-yl, 1H-indol-3-yl, 1H-indol-2-yl, 1H-indol-7-yl, 1-pyrroyl, 1H-benzotriazol-5-yl, 1H-benzoimidazol-5-yl, 2,3-dihydro-benzofuran-5-yl, 2,3-dihydro-isoindol-1-on-2-yl; benzo[1,2,3]thiadiazol-5-yl, benzo[1,2,3]thiadiazol-6-yl, benzothiazol-6-yl, benzothiazol-2-yl, and 4H-benzo[1,4]oxazin-3-on-7-yl, wherein Ar¹ is further optionally substituted with one or more groups selected from C₁₋₄alkyl, C₂₋₄alkenyl, C₁₋₄alkoxy, C₁₋₄alkenyloxy, phenoxy, 4-methoxyphenoxy, benzyl, acetoamino, methylsulfonyl, methoxycarbonyl, nitro, chloro, fluoro, bromo, iodo, 1-pyrroyl, 2-methyl-pyrro-1-yl, amino, phenylsulfonyl, aceto,1-piperidinyl, [1,2,3]thiadiazol-4-yl, 4-morpholinyl, methoxy, ethoxy, isopropyloxy, methythio, cyano, dimethylamino, hydroxy, methylaminosulfonyl, trifluoromethyl, trifluoromethoxy, phenyl, phenoxy, benzyl, 4-hydroxyl-phenyl, diethylamino, methylsulfonyl, aminosulfonyl, cyclohexyl, 1-pyrrolyl, 1H-pyrazol-3-yl, 5-tetrazolyl, 1-piperidinyl, 1-pyrazolyl, methylsulfonylmethyl, 3,5-dimethyl-pyrazolyl, pyrrolidin-2-on-1-yl; and

Q is selected from 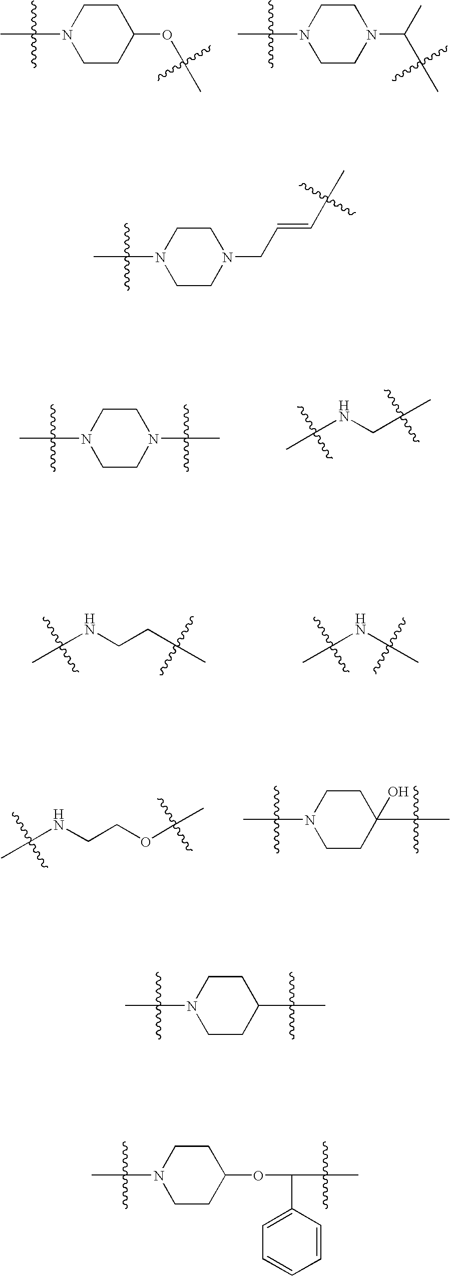 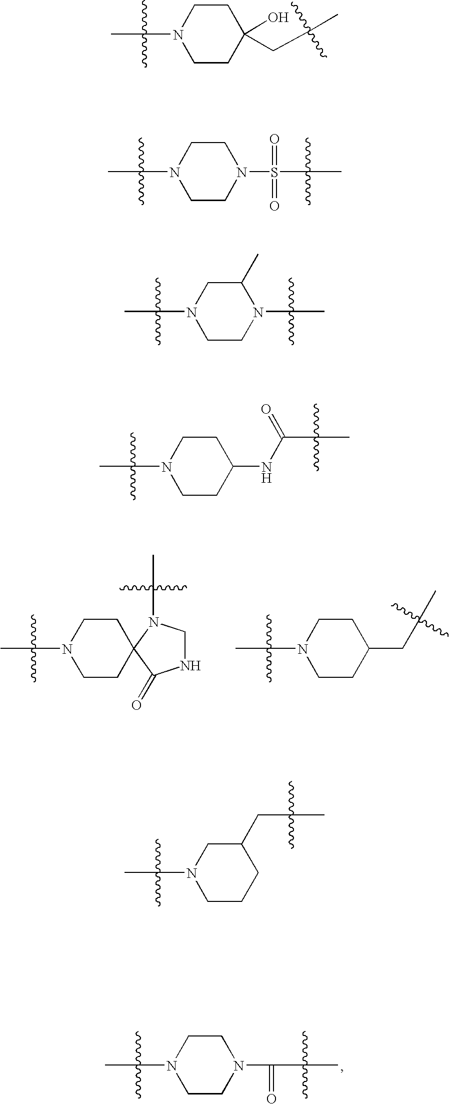
and wherein the left side nitrogen atom in the above-identified structures of Q is connected to the H in Ar¹-Q-H to form an amino group.

In a particular embodiment, the step of combining Ar¹-Q-H with 3-(1-piperidino)propylamine and a haloformate may be carried out at ambient temperature and in the presence of organic base such as diisopropylethylamine. The haloformate may be 4-nitrophenyl chloroformate.

Biological Evaluation

The compounds of the invention are found to be active towards H3 receptors in warm-blooded animal, e.g., human. Particularly the compounds of the invention are found to be effective H3 receptor ligands. In vitro assays, infra, demonstrate these surprising activities. These activities may be related to in vivo activity and may not be linearly correlated with binding affinity. In these in vitro assays, a compound is tested for their activity toward H3 receptors and pIC₅₀ is obtained to determine the activity for a particular compound towards H3 receptors.

FLIPR Assay Identification of Antagonists of the Human H3 Receptor

Cell Culture:

H3 receptor activation in response to histamine mediates intracellular Ca²⁺ mobilization in human H3 receptor transfected CHO-K1 cells. This increase in Ca²⁺ can be measured using the fluorometric imaging plate reader (FLIPR) employing Fluo-3AM loaded H3 receptor transfected cells. CHO-H3-Gα16 transfected cells were cultured in T225 cm² tissue culture flasks as monolayers in NUT Hams (with 1% (v/v) Glutamine) supplemented with 10% (v/v) heat inactivated fetal bovine serum and grown under 1 mg/ml. Geneticin antibiotic selection and 1 mg/ml Zeocin selection. Cultures were maintained at 37° C. in a humidified atmosphere of 5% CO₂ and passaged every 3 days.

Assay Buffer:

To 1000 mL of Hanks Balanced Salt solution, add 4.8 g of HEPES and 0.714 g probenecid (which is dissolved in 5 mL 1 M NaOH and added to the solution). This buffer is pH adjusted to 7.4 with NaOH. Assay Buffer contains 10% DMSO (v/v) was prepared for the compound preparation plates. Usually 200 ml (containing 20 ml neat DMSO) will be sufficient for 12×384 plates.

Loading Buffer:

To 120 mL Assay Buffer 100 mg BSA and 1 vial MDC FLIPR Calcium assay reagent (dissolved in assay buffer) was added immediately prior to loading cells:

Compound Vehicle Control Buffer:

400 μL DMSO was added to 20 mL Assay Buffer to produce 2% (v/v) solution (0.4% (v/v) final)

FLIPR Assay

Histamine EC50 determination: Cells were harvested using 1× dissociation solution and plated onto poly-D-lysine coated FLIPR plates at 1.0×10⁴ cells per well 18-24 hours prior to experiment. Media was removed from the cells by tipping and the plates gently blotted onto tissue to remove any excess medium. 30 μL loading buffer was added to all wells for 90 min at 37° C.

96 well histamine EC50 plate was made and then 40 μL was indexed into 4 quadrants in a 384 well plate. 96 well compound vehicle plates were made and indexed into a quadrant of a 384 well plate. Plates were transferred to FLIPR and run using a standard protocol. The results were used to calculate an EC50 for histamine.

Compound testing: Cells were harvested using 1× dissociation solution and plated onto poly-D-lysine coated FLIPR plates at 1.0×10⁴ cells per well 18-24 hours prior to experiment. Media was removed from the cells by tipping and the plates gently blotted onto tissue to remove any excess medium. 30 μL loading buffer was added to all wells for 90 min at 37° C. 96 well histamine plate (×10 EC50) was made and then 60 μL was indexed into 4 quadrants in a 384 well plate. Each 96 well compound plate was made and indexed into a quadrant of a 384 well plate. An ATP plate was made in a 96 well plate and then 60 μL was indexed into 4 quadrants in a 384 well plate. Plates transferred to FLIPR and run using a standard protocol. 30 μL of cells in loading buffer were placed in the wells of FLIPR 384 plate, 10 μL compound solution was added, values were read for 5 min to determine compound effects, 10 μL agonist solution was added, values were read to determine agonist response, 10 μL ATP added to and values were read for 5 min to determine ATP response.

Final assay concentrations: Compound concentration range=10 μM to 0.1 μM; Vehicle 0.4% DMSO; histamine=2× calculated EC50; ATP=1 μM

Assay for Inhibitors of [³H]-Histamine Binding to Human Recombinant H4 Receptor.

pIC₅₀ values were determined for compounds of the invention with a binding assay that allows the identification of inhibitors of [³H]-histamine by binding to membranes from CHO cells that over-express human recombinant H4 receptors. Cells suitable for performing this assay are commercially available, for example from Euroscreen as catalogue number 1220; [³H]-histamine suitable for performing this assay is commercially available, for example from Amersham as catalogue number TRK 631.

Compounds were dissolved in 500 μl of DMSO and diluted in DMSO to yield a 1 mM stock based on the formula weight of the compound. Stock solutions were diluted serially in DMSO in half log steps to give compound concentrations of 1000, 300, 100, 30 and 10 μM. Typically 5 point duplicate curves were determined. For 10 point curves single concentrations were typically 1000, 300, 100, 30, 10, 3, 1, 0.3, 0.1 and 0.03 μM. Assay buffer was added to each of the above concentrations to give 10% (v/v) DMSO (1:10 dilution). 5 μl of each diluted compound solution assayed in duplicate at a final compound concentration range of 10, 3, 1, 0.3 and 0.1 μM in 1% (v/v) DMSO. More active compounds were assayed at lower concentrations. Assays were performed in 96 deep well plates containing 0.1-10 μM compounds or 20 μM histamine; 0.015 mg protein/well H4 membranes and 3.9 nM of [³H]-histamine in a final volume of 200 μl. Plates were incubated at room temperature for 1.5 hours. The contents of the wells was captured on filters, washed 2×1 mL with Tris/EDTA wash buffer. The filters were dried for about 2 hrs at 60° C. and the [³H] determined by scintillation counting.

Data was analyzed to construct inhibition curves and pIC₅₀ estimated by non-linear regression using a 4 parameter logistic model. The IC₅₀ is the concentration of compound giving 50% inhibition relative to the plate controls. Thioperamide was used as the standard compound in this assay.
% Inhibition=100-((sample reading−NSB reading)/(control reading−NSB reading)×100)
pIC₅=−log(IC₅₀)

### EXAMPLES

The invention will further be described in more detail by the following Examples which describe methods whereby compounds of the present invention may be prepared, purified, analyzed and biologically tested, and which are not to be construed as limiting the invention.

Abbreviation used in the Following Examples and General Process Conditions:

aq.: aqueous;

atm: atmospheric pressure;

BOC: 1,1-dimethylethoxycarbonyl;

ACN: acetonitrile;

DCM: dichloromethane;

DMR: N,N-dimethylformamide;

DMSO: dimethyl sulfoxide;

EtOH: ethanol;

Et₂O: diethyl ether;

EtOAc: ethyl acetate;

h: hour(s);

HPLC: high performance liquid chromatography;

EDC-HCl: 1-(3-Dimethylaminopropyl)-3-ethylcarbodiimide hydrochloride;

HOBT: 1-hydroxybenzotriazole;

MeOH: methanol;

min: minutes;

MS: mass spectrum;

NMR: nuclear magnetic resonance;

psi: pounds per square inch;

RT: room temperature;

sat.: saturated;

TEA: triethylamine;

TFA: trifluoroacetic acid;

THF: tetrahydrofuran.

Temperatures are given in degrees Celsius (° C.). Unless otherwise stated, operations were carried out at room or ambient temperature (18-25° C.).

Chromatography means flash column chromatography on silica gel unless otherwise noted. Solvent mixture compositions are given as volume percentages or volume ratios.

Where noted that a final compound was converted to the citrate salt, the free base was dissolved in MeOH, DCM, or ACN, combined with citric acid (1.0 equivalents) in MeOH, concentrated under reduced pressure and dried under vacuum (25-60° C.). When indicated that the salt was isolated by filtration from Et₂O, the citrate salt of the compound was stirred in Et₂O for 4-18 h, recovered by filtration, washed with Et₂O, and dried under vacuum (25-60° C.).

### Example 1

### N-(3,4-dichlorobenzyl)-4-methylpiperazine-1-carboxamide

To a solution of 3,4-dichlorobenzylamine (0.195 g, 1.11 mmol) and diisopropylethylamine (0.193 mL, 1.11 mmol) in 4 mL of THF was added a preformed solution of 4-nitrophenyl chloroformate (0.223 g, 1.11 mmol) in 4 mL of TBF. The reaction mixture was stirred at RT for 3.5 h. To this solution was added N-methyl piperazine (0.442 g, 4.42 mmol) and the resulting solution was stirred at RT for 16 h. The reaction mixture was concentrated under reduced pressure, diluted with EtOAc (50 mL) and the solution was washed with saturated aqueous sodium bicarbonate (2×50 mL) and brine (50 mL). The solvent was removed under reduced pressure and the residue was subjected to supercritical fluid chromatography (21 mm×150 mm diol-bonded SiO₂ (6 μm particle size), isocratic method, 25% MeOH (containing 0.5% isopropyl amine) in CO₂) to afford the title compound as a pale yellow solid (0.245 g, 73%). MS m/z 302.2 (M+H)⁺; ¹H NMR (300.1 MHz, DMSO-d₆) δ 2.17 (s, 3H), 2.22-2.26 (m, 4H), 3.25-3.31 (m, 4H), 4.20 (d, J=5.8 Hz, 2H), 7.12 (t, J=5.8 Hz, 1H), 7.23 (dd, J=8.2, 2.0 Hz, 1H), 7.47 (d, J=1.8 Hz, 1H), 7.56 (d, J=8.2 Hz, 1H).

The above procedure may be used to synthesize the following compounds:

| Example | | | | Exact | |
|---|---|---|---|---|---|
| No. | Structure | Name | MW | Mass | MH+ |
| 1. | 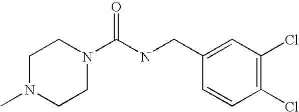 | N-(3,4-dichlorobenzyl)-4- methylpiperazine-1-carboxamide | 302.2 | 301.1 | 302.1 |
| 2. | 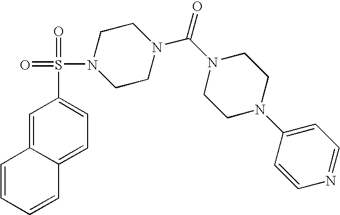 | (4-(napthalen-2- ylsulfonyl)piperazin-1-yl)(4-(pyridin- 4-yl)piperzin-1-yl)methanone | 465.58 | 465.2 | 466.2 |
| 3. | 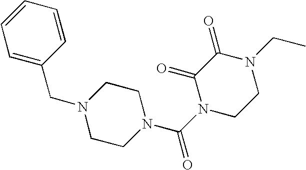 | 1-(4-benzylpiperazine-1-carbonyl)- 4-ethylpiperazine-2,3-dione | 344.41 | 344.2 | 345.2 |
| 4. | 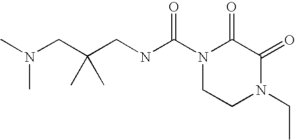 | N-(3-(dimethylamino)-2,2- dimethylpropyl)-4-ethyl-2,3- dioxopiperazine-1-carboxamide | 298.38 | 298.2 | 299.2 |
| 5. | 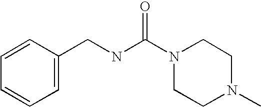 | N-benzyl-4-methylpiperazine-1- carboxamide | 233.31 | 233.2 | 234.2 |
| 6. | 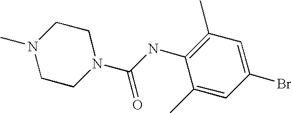 | N-(4-bromo-2,6-dimethylphenyl)-4- methylpiperazine-1-carboxamide | 326.24 | 325.1 | 326.1 |
| 7. | 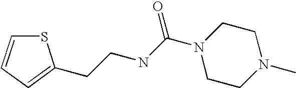 | 4-methyl-N-(2-(thiophen-2- yl)ethyl)piperazine-1-carboxamide | 253.37 | 253.1 | 254.1 |
| 8. | 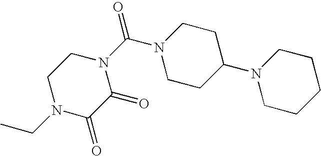 | 1-(1,4′-bipiperidine-1′-carbonyl)-4- ethylpiperazine-2,3-dione | 336.43 | 336.2 | 337.2 |
| 9. | 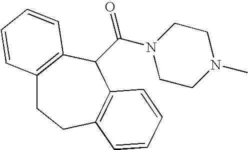 | 5-[(4-methylpiperazin-1- yl)carbonyl]-10,11-dihydro-5H- dibenzo[b,f]azepine | 321.42 | 321.2 | 322.2 |
| 10. | 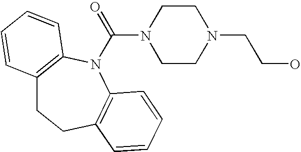 | 2-[4-(10,11-dihydro-5H- dibenzo[b,f]azepin-5- ylcarbonyl)piperazin-1-yl]ethanol | 351.45 | 351.2 | 352.2 |
| 11. | 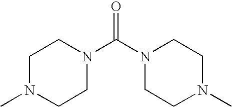 | bis(4-methylpiperazin-1- yl)methanone | 226.32 | 226.2 | 227.2 |
| 12. | 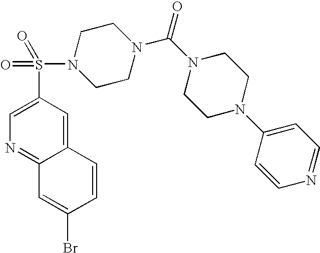 | (4-(7-bromoquinolin-3- ylsulfonyl)piperazin-1-yl)(4-pyridin- 4-yl)piperazin-1-yl)methanone | 545.46 | 544.1 | 545.1 |
| 13. | 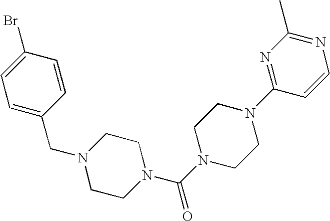 | (4-(4-bromobenzyl)piperazin-1- yl)(4-(2-methylpyrimidin-4- yl)piperazin-1-yl)methanone | 459.39 | 458.1 | 459.2 |
| 14. | 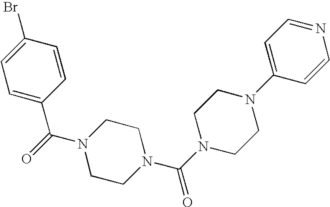 | (4-bromophenyl)(4-(4-(pyridin-4- yl)piperazine-1-carbonyl)piperazin- 1-yl)methanone | 458.36 | 457.1 | 458.1 |
| 15. | 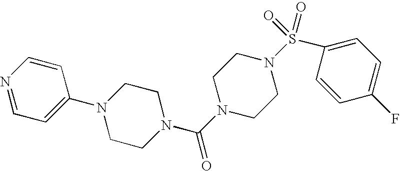 | (4-(4- fluorophenylsulfonyl)piperazin-1- yl)(4-(pyridin-4-yl)piperazin-1- yl)methanone | 433.51 | 433.2 | 434.2 |
| 16. | 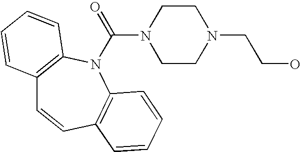 | 2-[4-(5H-dibenzo[b,f]azepin-5- ylcarbonyl)piperazin-1-yl]ethanol | 349.43 | 349.2 | 350.2 |
| 17. | 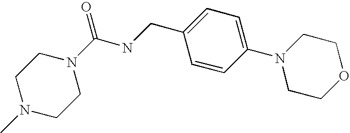 | 4-methyl-N-(4- morphonlinobenzyl)piperazine-1- carboxamide | 318.42 | 318.2 | 319.2 |
| 18. | 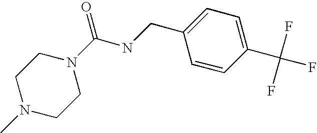 | 4-methyl-N-(4- (trifluoromethyl)benzyl)piperazine- 1-carboxamide | 301.31 | 301.1 | 302.1 |
| 19. | 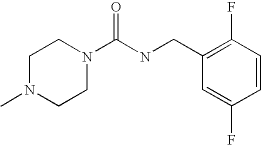 | N-(2,5-difluorobenzyl)-4- methylpiperazine-1-carboxamide | 269.29 | 269.1 | 270.1 |
| 20. | 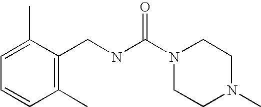 | N-(2,6-dimethylbenzyl)-4- methylpiperazine-1-carboxamide | 261.37 | 261.2 | 262.2 |
| 21. | 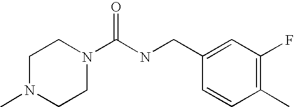 | N-(3-fluoro-4-methylbenzyl)-4- methylpiperazine-1-carboxamide | 265.33 | 265.2 | 266.2 |
| 22. | 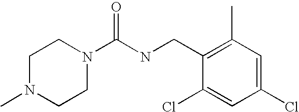 | N-(2,4-dichloro-6-methylbenzyl)-4- methylpiperazine-1-carboxamide | 316.23 | 315.1 | 316.1 |
| 23. | 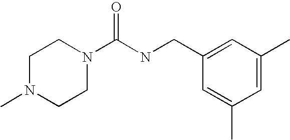 | N-(3,5-dimethylbenzyl)-4- methylpiperazine-1-carboxamide | 261.37 | 261.2 | 262.2 |
| 24. | 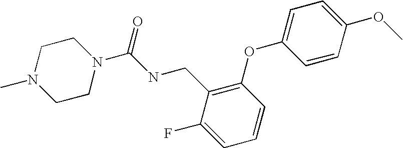 | N-(2-fluoro-6-(4- methoxyphenoxy)benzyl)-4- methylpiperazine-1-carboxamide | 373.43 | 373.2 | 374.2 |
| 25. | 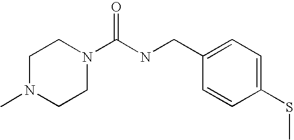 | 4-methyl-N-(4- (methylthio)benzyl)piperazine-1- carboxamide | 279.41 | 279.1 | 280.1 |
| 26. | 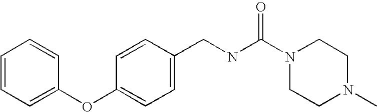 | 4-methyl-N-(4- phenoxybenzyl)piperazine-1- carboxamide | 325.41 | 325.2 | 326.2 |
| 27. | 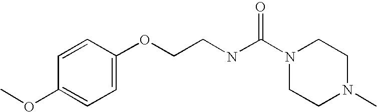 | N-(2-(4-methoxyphenoxy)ethyl)-4- methylpiperazine-1-carboxamide | 293.37 | 293.2 | 294.2 |
| 28. | 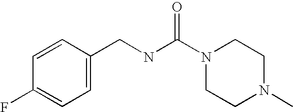 | N-(4-fluorobenzyl)-4- methylpiperazine-1-carboxamide | 251.3 | 251.1 | 252.2 |
| 29. | 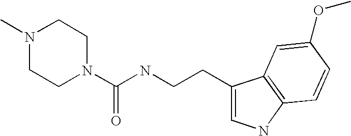 | N-(2-(5-methoxy-1H-indol-3- yl)ethyl)-4-methylpiperazine-1- carboxamide | 316.4 | 316.2 | 317.2 |
| 30. | 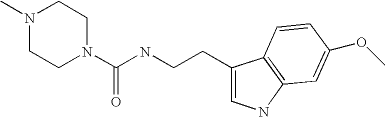 | N-(2-(6-methoxy-1H-indol-3- yl)ethyl)-4-methylpiperazine-1- carboxamide | 316.4 | 316.2 | 317.2 |
| 31. | 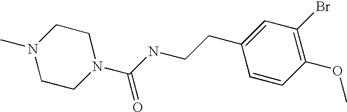 | N-(3-bromo-4-methoxyphenethyl)- 4-methylpiperazine-1-carboxamide | 356.26 | 355.1 | 356.1 |
| 32. | 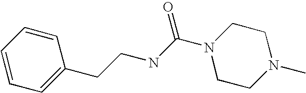 | 4-methyl-N-phenethylpiperazine-1- carboxamide | 247.34 | 247.2 | 248.2 |
| 33. | 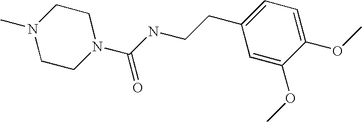 | N-(3,4-dimethoxyphenethyl)-4- methylpiperazine-1-carboxamide | 307.39 | 307.2 | 308.2 |
| 34. | 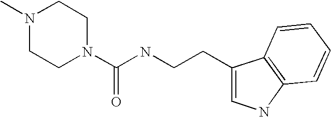 | N-(2-(1H-indol-3-yl)ethyl)-4- methylpiperazine-1-carboxamide | 286.38 | 286.2 | 287.2 |
| 35. | 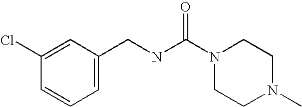 | N-(3-chlorobenzyl)-4- methylpiperazine-1-carboxamide | 267.76 | 267.1 | 268.1 |
| 36. | 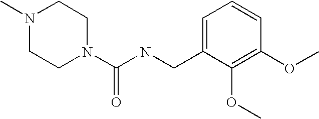 | N-(2,3-dimethoxybenzyl)-4- methylpiperazine-1-carboxamide | 293.37 | 293.2 | 294.2 |
| 37. | 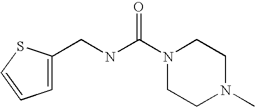 | 4-methyl-N-(thiophen-2- ylmethyl)piperazine-1-carboxamide | 239.34 | 239.1 | 240.1 |
| 38. | 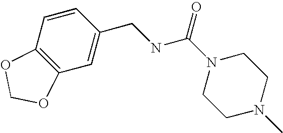 | N-(benzo[d][1,3]dioxol-5-ylmethyl)- 4-methylpiperazine-1-carboxamide | 277.32 | 277.1 | 278.2 |
| 39. | 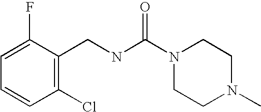 | N-(2-chloro-6-fluorobenzyl)-4- methylpiperazine-1-carboxamide | 287.75 | 285.1 | 286.1 |
| 40. | 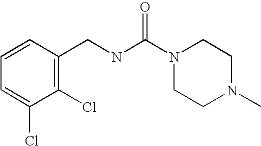 | N-(2,3-dichlorobenzyl)-4- methylpiperazine-1-carboxamide | 302.2 | 301.1 | 302.1 |
| 41. | 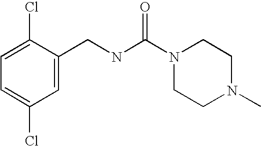 | N-(2,5-dichlorobenzyl)-4- methylpiperazine-1-carboxamide | 302.2 | 301.1 | 302.1 |
| 42. | 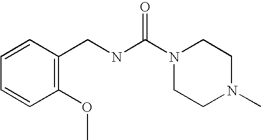 | N-(2-methoxybenzyl)-4- methylpiperazine-1-carboxamide | 263.34 | 263.2 | 264.2 |
| 43. | 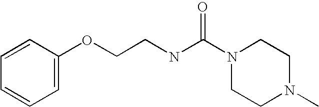 | 4-methyl-N-(2- phenoxyethyl)piperazine-1- carboxamide | 263.34 | 263.2 | 264.2 |
| 44. | 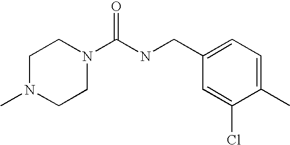 | N-(3-chloro-4-methylbenzyl)-4- methylpiperazine-1-carboxamide | 281.79 | 281.1 | 282.1 |
| 45. | 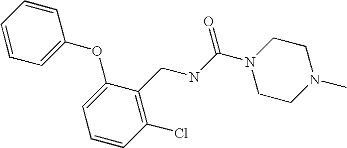 | N-(2-chloro-6-phenoxybenzyl)-4- methylpiperazine-1-carboxamide | 359.86 | 359.1 | 360.1 |
| 46. | 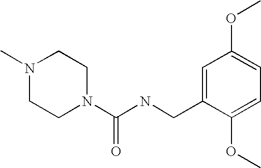 | N-(2,5-dimethoxybenzyl)-4- methylpiperazine-1-carboxamide | 293.37 | 293.2 | 294.2 |
| 47. | 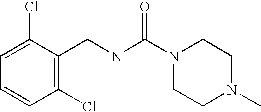 | N-(2,6-dichlorobenzyl)-4- methylpiperazine-1-carboxamide | 302.2 | 301.1 | 302.1 |
| 48. | 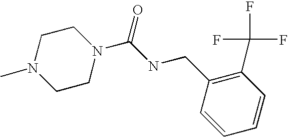 | 4-methyl-N-(2- (trifluoromethyl)benzyl)piperazine- 1-carboxamide | 301.31 | 301.1 | 302.1 |
| 49. | 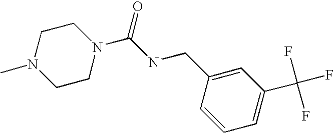 | 4-methyl-N-(3- (trifluoromethyl)benzyl)piperazine- 1-carboxamide | 301.31 | 301.1 | 302.1 |
| 50. | 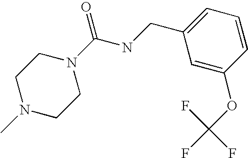 | 4-methyl-N-(3- (trifluoromethoxy)benzyl)piperazine- 1-carboxamide | 317.31 | 317.1 | 318.1 |
| 51. | 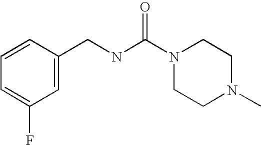 | N-(3-fluorobenzyl)-4- methylpiperazine-1-carboxamide | 251.3 | 251.1 | 252.2 |
| 52. | 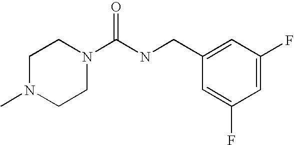 | N-(3,5-difluorobenzyl)-4- methylpiperazine-1-carboxamide | 269.29 | 269.1 | 270.1 |
| 53. | 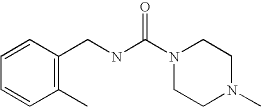 | 4-methyl-N-(2- methylbenzyl)piperazine-1- carboxamide | 247.34 | 247.2 | 248.2 |
| 54. | 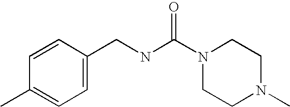 | 4-methyl-N-(4- methylbenzyl)piperazine-1- carboxamide | 247.34 | 247.2 | 248.2 |
| 55. | 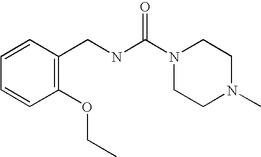 | N-(2-ethoxybenzyl)-4- methylpiperazine-1-carboxamide | 277.37 | 277.2 | 278.2 |
| 56. | 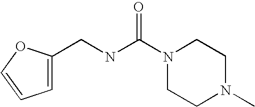 | N-(furan-2-ylmethyl)-4- methylpiperazine-1-carboxamide | 223.27 | 223.1 | 224.1 |
| 57. | 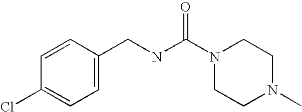 | N-(4-chlorobenzyl)-4- methylpiperazine-1-carboxamide | 267.76 | 267.1 | 268.1 |
| 58. | 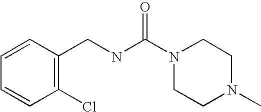 | N-(2-chlorobenzyl)-4- methylpiperazine-1-carboxamide | 267.76 | 267.1 | 268.1 |
| 59. | 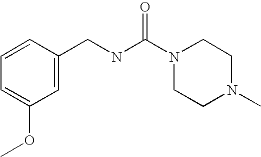 | N-(3-methoxybenzyl)-4- methylpiperazine-1-carboxamide | 263.34 | 263.2 | 264.2 |
| 60. | 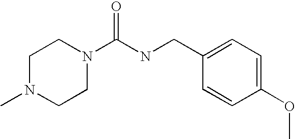 | N-(4-methoxybenzyl)-4- methylpiperazine-1-carboxamide | 263.34 | 263.2 | 264.2 |
| 61. | 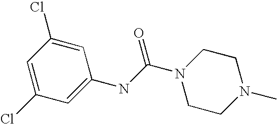 | N-(3,5-dichlorophenyl)-4- methylpiperazine-1-carboxamide | 288.18 | 287.1 | 288.1 |
| 62. | 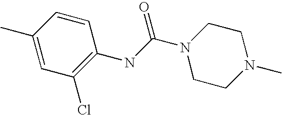 | N-(2-chloro-4-methylphenyl)-4- methylpiperazine-1-carboxamide | 267.76 | 267.1 | 268.1 |
| 63. | 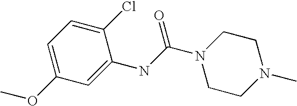 | N-(2-chloro-5-methoxyphenyl)-4- methylpiperazine-1-carboxamide | 283.76 | 283.1 | 284.1 |
| 64. | 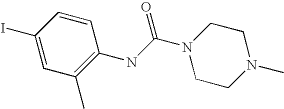 | N-(4-iodo-2-methylphenyl)-4- methylpiperazine-1-carboxamide | 359.21 | 359.0 | 360.1 |
| 65. | 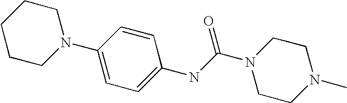 | 4-methyl-N-(4-piperidin-1- yl)phenyl)piperazine-1- carboxamide | 302.42 | 302.2 | 303.2 |
| 66. | 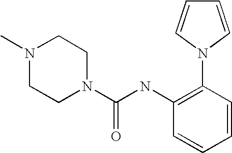 | N-(2-(1H-pyrrol-1-yl)phenyl)-4- methylpiperazine-1-carboxamide | 284.36 | 284.2 | 285.2 |
| 67. | 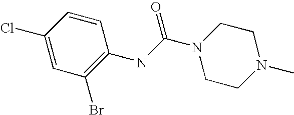 | N-(2-bromo-4-chlorophenyl)-4- methylpiperazine-1-carboxamide | 332.63 | 331.0 | 332.0 |
| 68. | 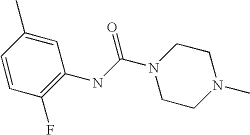 | N-(2-fluoro-5-methylphenyl)-4- methylpiperazine-1-carboxamide | 251.3 | 251.1 | 252.2 |
| 69. | 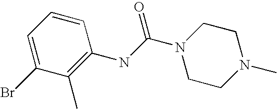 | N-(3-bromo-2-methylphenyl)-4- methylpiperazine-1-carboxamide | 312.21 | 311.1 | 312.1 |
| 70. | 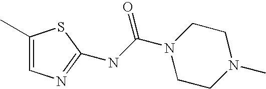 | 4-methyl-N-(5-methylthiazol-2- yl)piperazine-1-carboxamide | 240.33 | 240.1 | 241.1 |
| 71. | 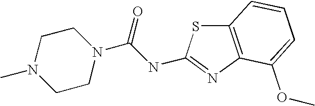 | N-(4-methoxybenzo[d]thiazol-2-yl)- 4-methylpiperazine-1-carboxamide | 306.39 | 306.1 | 307.1 |
| 72. | 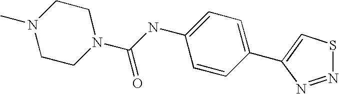 | N-(4-(1,2,3-thiadiazol-4-yl)phenyl)- 4-methylpiperazine-1-carboxamide | 303.39 | 303.1 | 304.1 |
| 73. | 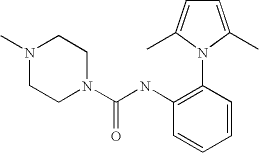 | N-(2-(2,5-dimethyl-1H-pyrrol-1- yl)phenyl)-4-methylpiperazine-1- carboxamide | 312.42 | 312.2 | 313.2 |
| 74. | 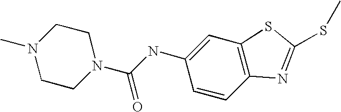 | 4-methyl-N-(2- (methylthio)benzo[d]thiazol-6- yl)piperazine-1-carboxamide | 322.46 | 322.1 | 323.1 |
| 75. | 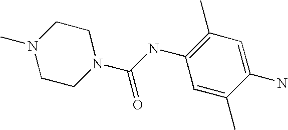 | N-(4-amino-2,5-dimethylphenyl)-4- methylpiperazine-1-carboxamide | 262.36 | 262.2 | 263.2 |
| 76. | 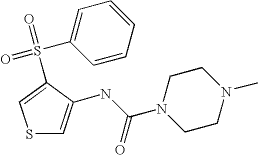 | 4-methyl-N-(4- (phenylsulfonyl)thiophen-3- yl)piperazine-1-carboxamide | 365.48 | 365.1 | 366.1 |
| 77. | 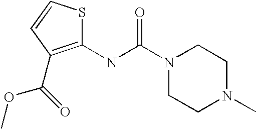 | methyl 2-{[(4-methylpiperazin-1- yl)carbonyl]amino}thiophene-3- carboxylate | 283.35 | 283.1 | 284.1 |
| 78. | 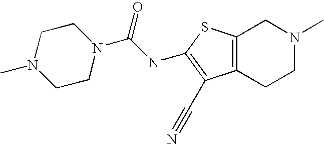 | N-(3-cyano-6-methyl-4,5,6,7- tetrahydrothieno[2,3-c]pyridin-2-yl)- 4-methylpiperazine-1-carboxamide | 319.43 | 319.1 | 320.2 |
| 79. | 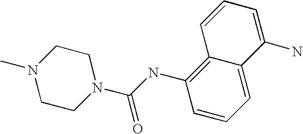 | N-(5-aminonaphthalen-1-yl)-4- methylpiperazine-1-carboxamide | 284.36 | 284.2 | 285.2 |
| 80. | 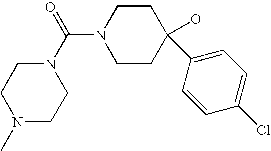 | (4-(4-chlorophenyl)-4- hydroxypiperidin-1-yl)(4- methylpiperazin-1-yl)methanone | 337.85 | 337.2 | 338.2 |
| 81. | 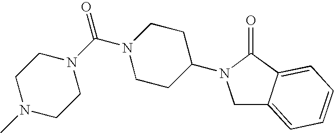 | 2-(1-(4-methylpiperazine-1- carbonyl)piperidin-4-yl)isoindolin-1- one | 342.44 | 342.2 | 343.2 |
| 82. | 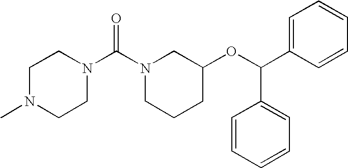 | (3-(benzhydryloxy)piperidin-1-yl)(4- methylpiperazin-1-yl)methanone | 393.53 | 393.2 | 394.2 |
| 83. | 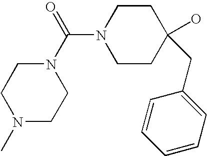 | (4-benzyl-4-hydroxypiperidin-1- yl)(4-methylpiperazin-1- yl)methanone | 317.43 | 317.2 | 318.2 |
| 84. | 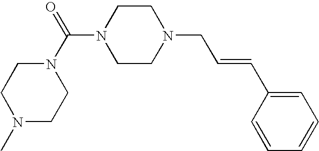 | (E)-(4-cinnamylpiperazin-1-yl)(4- methylpiperazin-1-yl)methanone | 328.46 | 328.2 | 329.2 |
| 85. | 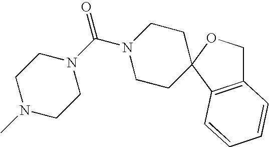 | (4-methylpiperazin-1-yl)(3H- spiro[isobenzofuran-1,4′- piperidine]-1′-yl)methanone | 315.41 | 315.2 | 316.2 |
| 86. | 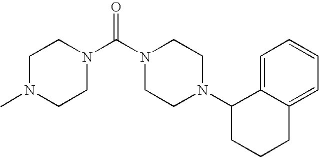 | (4-methylpiperazin-1-yl)(4-(1,2,3,4- tetrahydronaphthalen-1- yl)piperazin]-1-yl)methanone | 342.48 | 342.2 | 343.2 |
| 87. | 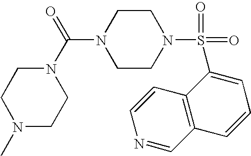 | (4-(isoquinolin-5- ylsulfonly)piperazin-1-yl)(4- methylpiperazin-1-yl)methanone | 403.5 | 403.2 | 404.2 |
| 88. | 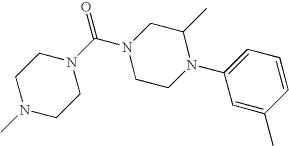 | (3-methyl-4-m-tolylpiperazin-1- yl)(4-methylpiperazin-1- yl)methanone | 316.45 | 316.2 | 317.2 |
| 89. | 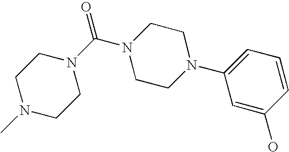 | (4-(3-hydroxyphenyl)piperazin-1- yl)(4-methylpiperazin-1- yl)methanone | 304.39 | 304.2 | 305.2 |
| 90. | 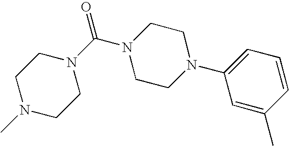 | (4-methylpiperazin-1-yl)(4-m- tolylpiperazin-1-yl)methanone | 302.42 | 302.2 | 303.2 |
| 91. | 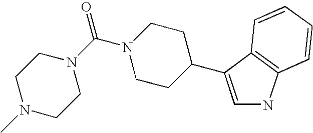 | (4-(1H-indol-3-yl)piperidin-1-yl)(4- methylpiperazin-1-yl)methanone | 326.44 | 326.2 | 327.2 |
| 92. | 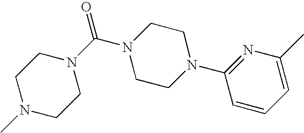 | (4-methylpiperazin-1-yl)(4-(6- methylpyridin-2-yl)piperazin-1- yl)methanone | 303.41 | 303.2 | 304.2 |
| 93. | 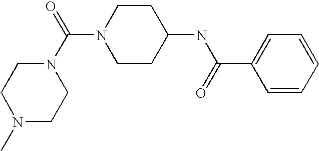 | N-(1-(4-methylpiperazine-1- carbonyl)piperidin-4-yl)benzamide | 330.43 | 330.2 | 331.2 |
| 94. | 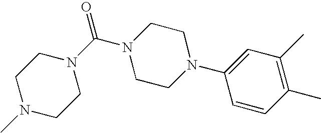 | (4-(3,4-dimethylphenyl)piperazin-1- yl)(4-methylpiperazin-1- yl)methanone | 316.45 | 316.2 | 317.2 |
| 95. | 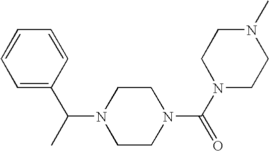 | (4-methylpiperazin-1-yl)(4-(1- phenylethyl)piperazin-1- yl)methanone | 316.45 | 316.2 | 317.2 |
| 96. | 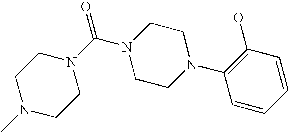 | (4-(2-hydroxyphenyl)piperazin-1- yl)(4-methylpiperazin-1- yl)methanone | 304.39 | 304.2 | 305.2 |
| 97. | 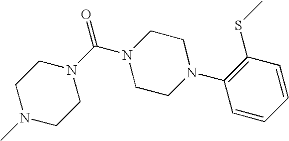 | (4-methylpiperazin-1-yl)(4-(2- (methylthio)phenyl)piperazin-1- yl)methanone | 334.49 | 334.2 | 335.2 |
| 98. | 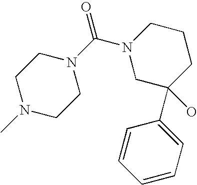 | (3-hydroxy-3-phenylpiperidin-1- yl)(4-methylpiperazin-1- yl)methanone | 303.4 | 303.2 | 304.2 |
| 99. | 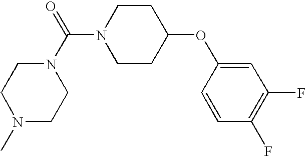 | (4-(3,4-difluorophenoxy)piperidin-1- yl)(4-methylpiperazin-1- yl)methanone | 339.38 | 339.2 | 340.2 |
| 100. | 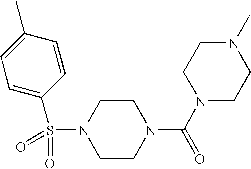 | (4-methylpiperazin-1-yl)(4- tosylpiperazin-1-yl)methanone | 366.48 | 366.2 | 367.2 |
| 101. | 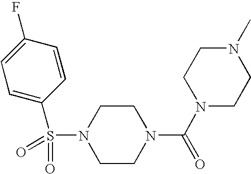 | (4-(4- fluorophenylsulfonyl)piperazin-1- yl)(4-methylpiperazin-1- yl)methanone | 370.45 | 370.1 | 371.2 |
| 102. | 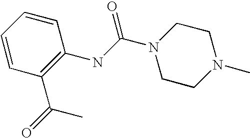 | N-(2-ethanoylphenyl)-4- methylpiperazine-1-carboxamide | 261.32 | 261.1 | 262.2 |
| 103. | 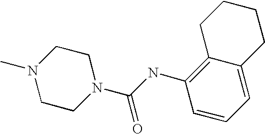 | 4-methyl-N-(5,6,7,8- tetrahydronaphthalen-1- yl)piperazine-1-carboxamide | 273.38 | 273.2 | 274.2 |
| 104. | 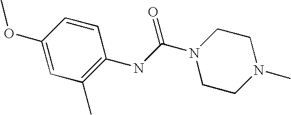 | N-(4-methoxy-2-methylphenyl)-4- methylpiperazine-1-carboxamide | 263.34 | 263.2 | 264.2 |
| 105. | 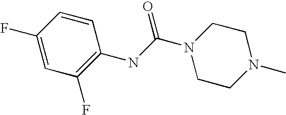 | N-(2,4-diflurophenyl)-4- methylpiperazine-1-carboxamide | 255.27 | 255.1 | 256.1 |
| 106. | 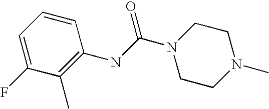 | N-(3-fluoro-2-methylphenyl)-4- methylpiperazine-1-carboxamide | 251.3 | 251.1 | 252.2 |
| 107. | 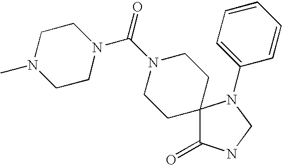 | 8-(4-methylpiperazine-1-carbonyl)- 1-phenyl-1,3,8- triazaspiro[4.5]decan-4-one | 357.46 | 357.2 | 358.2 |
| 108. | 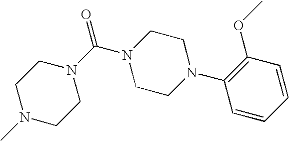 | (4-(2-methoxyphenyl)piperazin-1- yl)(4-methylpiperazin-1- yl)methanone | 318.42 | 318.2 | 319.2 |
| 109. | 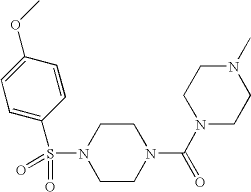 | (4-(4- methoxyphenylsulfonyl)piperazin-1- yl)(4-methylpiperazin-1- yl)methanone | 382.48 | 382.2 | 383.2 |
| 110. | 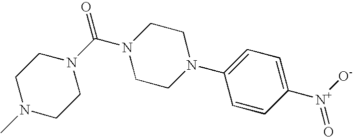 | (4-methylpiperazin-1-yl)(4-(4- nitrophenyl)piperazin-1- yl)methanone | 333.39 | 333.2 | 334.2 |
| 111. | 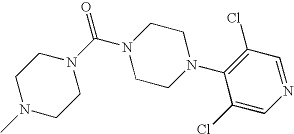 | (4-(3,5-dichloropyridin-4- yl)piperazin-1-yl)(4- methylpiperazin-1-yl)methanone | 358.27 | 357.1 | 358.1 |
| 112. | 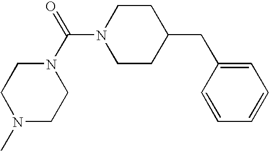 | (4-benzylpiperidin-1-yl)(4- methylpiperazin-1-yl)methanone | 301.43 | 301.2 | 302.2 |
| 113. | 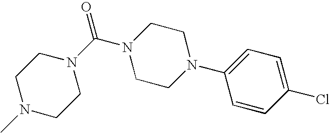 | (4-(4-chlorophenyl)piperazin-1- yl)(4-methylpiperazin-1- yl)methanone | 322.84 | 322.2 | 323.2 |
| 114. | 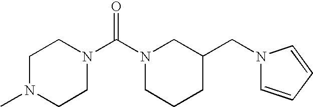 | (3-((1H-pyrrol-1-yl)methylpiperidin- 1-yl)(4-methylpiperazin-1- yl)methanone | 290.41 | 290.2 | 291.2 |
| 115. | 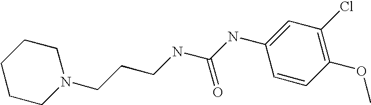 | 1-(3-chloro-4-methoxyphenyl)-3-(3- (piperidin-1-yl)propyl)urea | 325.84 | 325.2 | 326.2 |
| 116. | 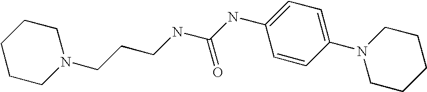 | 1-(4-(piperidin-1-yl)phenyl)-3-(3- (piperidin-1-yl)propyl)urea | 344.5 | 344.3 | 345.3 |
| 117. | 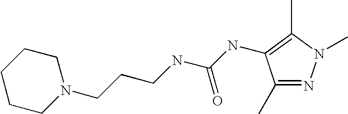 | 1-(3-(piperidin-1-yl)propyl)-3-(1,3,5- trimethyl-1H-pyrazol-4-yl)urea | 293.41 | 293.2 | 294.2 |
| 118. | 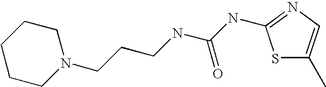 | 1-(5-methylthiazol-2-yl)-3-(3- (piperidin-1-yl)propyl)urea | 282.41 | 282.2 | 283.2 |
| 119. | 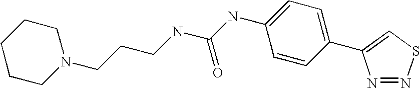 | (1-(4-(1,2,3-thiadiazol-4-yl)phenyl)- 3-(3-(piperidin-1-yl)propyl)urea | 345.47 | 345.2 | 346.2 |
| 120. | 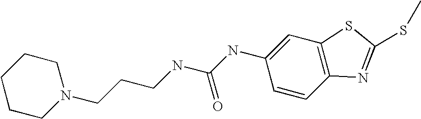 | 1-(2-(methylthio)benzo[d]thiazol-6- yl)-3-(3-(piperidin-1-yl)propyl)urea | 364.54 | 364.1 | 365.1 |
| 121. | 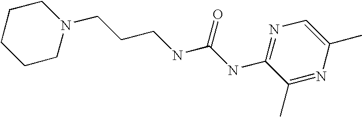 | 1-(3,5-dimethylpyrazin-2-yl)-3-(3- (piperidin-1-yl)propyl)urea | 291.4 | 291.2 | 292.2 |
| 122. | 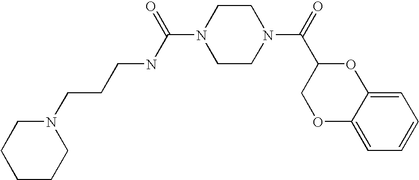 | 4-(2,3-dihydrobenzo[b][1,4]dioxine- 2-carbonyl)-N-(3-(piperidin-1- yl)propyl)piperazine-1-carboxamide | 416.52 | 416.2 | 417.3 |
| 123. | 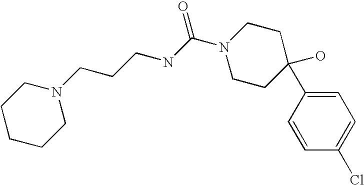 | 4-(4-chlorophenyl)-4-hydroxy-N-(3- (piperidin-1-yl)propyl)piperidine-1- carboxamide | 379.93 | 379.2 | 380.2 |
| 124. | 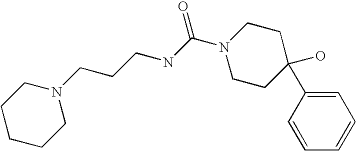 | 4-hydroxy-4-phenyl)-N-(3-(piperidin- 1-yl)propyl)piperidine-1- carboxamide | 345.48 | 345.2 | 346.2 |
| 125. | 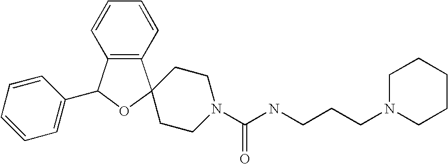 | 3-phenyl-N-(3-(piperidin-1- yl)propyl)-3H-spiro[isobenzofuran- 1,4′-piperidine]-1′-carboxamide | 433.59 | 433.3 | 434.3 |
| 126. | 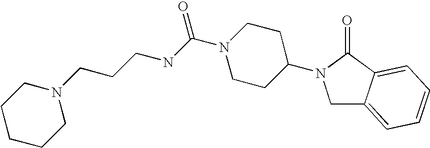 | 4-(1-oxoisoindolin-2-yl)-N-(3- (piperidin-1-yl)propyl)piperidine-1- carboxamide | 384.52 | 384.3 | 385.3 |
| 127. | 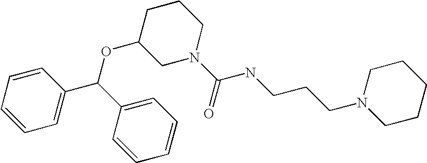 | 3-(benzhydryloxy)-N-(3-(piperidin- 1-yl)propyl)piperidine-1- carboxamide | 435.61 | 435.3 | 436.3 |
| 128. | 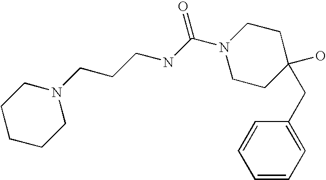 | 4-benzyl-4-hydroxy-N-(3-(piperidin- 1-yl)propyl)piperidine-1- carboxamide | 359.51 | 359.3 | 360.3 |
| 129. | 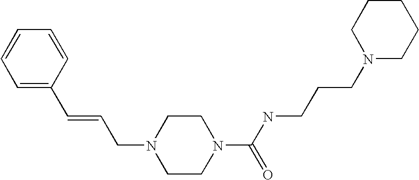 | 4-cinnamyl-N-(3-(piperidin-1- yl)propyl)piperazine-1-carboxamide | 370.54 | 370.3 | 371.3 |
| 130. | 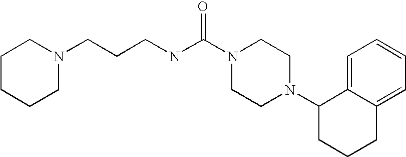 | N-(3-(piperidin-1-yl)propyl)-4- (1,2,3,4-tetrahydronaphthalen-1- yl)piperazine-1-carboxamide | 384.56 | 384.3 | 385.3 |
| 131. | 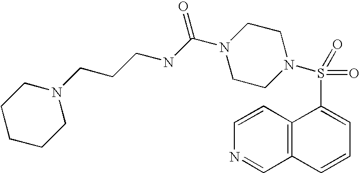 | 4-(isoquinolin-5-ylsulfonyl)-N-(3- (piperidin-1-yl)propyl)piperazine-1- carboxamide | 445.59 | 445.2 | 446.2 |
| 132. | 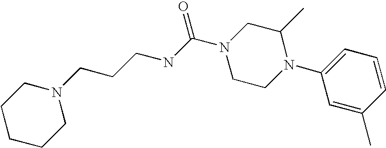 | 3-methyl-N-(3-(piperidin-1- yl)propyl)-4-m-tolylpiperazine-1- carboxamide | 358.53 | 358.3 | 359.3 |
| 133. | 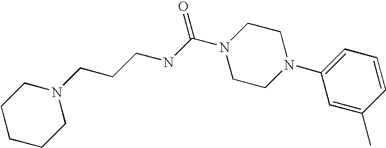 | N-(3-(piperidin-1-yl)propyl)-4-m- tolylpiperazine-1-carboxamide | 344.5 | 344.3 | 345.3 |
| 134. | 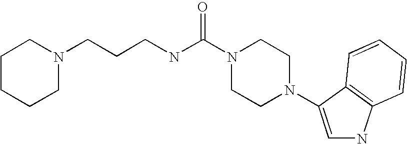 | 4-(1H-indol-3-yl)-N-(3-(piperidin-1- yl)propyl)piperazine-1-carboxamide | 368.52 | 368.3 | 369.3 |
| 135. | 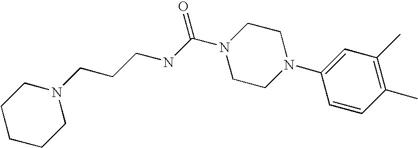 | 4-(3,4-dimethylphenyl)-N-(3- (piperidin-1-yl)propyl)piperazine-1- carboxamide | 358.53 | 358.3 | 359.3 |
| 136. | 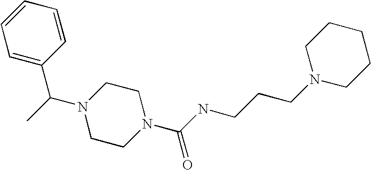 | 4-(1-phenylethyl)-N-(3-(piperidin-1- yl)propyl)piperazine-1-carboxamide | 358.53 | 358.3 | 359.3 |
| 137. | 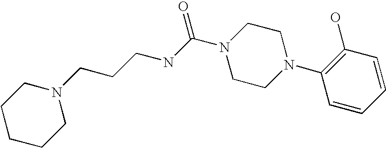 | 4-(2-hydroxyphenyl)-N-(3- (piperidin-1-yl)propyl)piperazine-1- carboxamide | 346.47 | 346.2 | 347.2 |
| 138. | 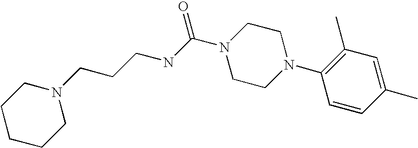 | 4-(2,4-dimethylphenyl)-N-(3- (piperidin-1-yl)propyl)piperazine-1- carboxamide | 358.53 | 358.3 | 359.3 |
| 139. | 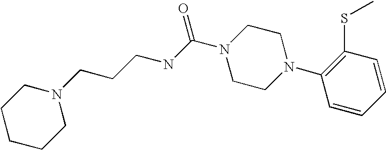 | 4-(2-(methylthio)phenyl)-N-(3- (piperidin-1-yl)propyl)piperazine-1- carboxamide | 376.57 | 376.2 | 377.2 |
| 140. | 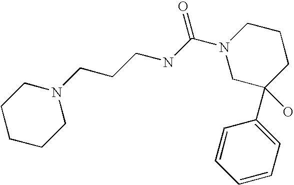 | 3-hydroxy-3-phenyl-N-(3-(piperidin- 1-yl)propyl)piperidine-1- carboxamide | 345.48 | 345.2 | 346.2 |
| 141. | 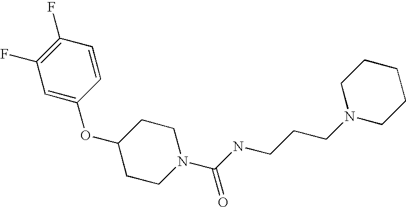 | 4-(3,4-diflurophenoxy)-N-(3- (piperidin-1-yl)propyl)piperidine-1- carboxamide | 381.46 | 381.2 | 382.2 |
| 142. | 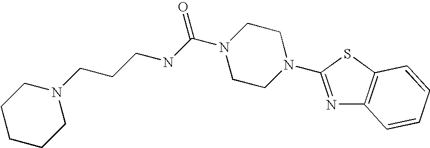 | 4-(benzo[d]thiazol-2-yl)-N-(3- (piperidin-1-yl)propyl)piperazine-1- carboxamide | 387.55 | 387.2 | 388.2 |
| 143. | 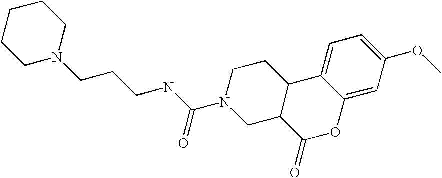 | 8-methoxy-5-oxo-N-(3-piperidin-1- ylpropyl)-1,4a,5,10b-tetrahydro-2H- chromeno[3,4-c]pyridine-3(4H)- carboxamide | 401.5 | 401.2 | 402.2 |
| 144. | 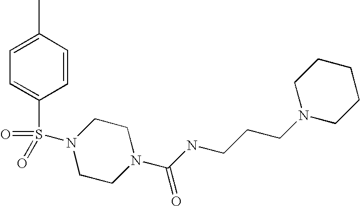 | N-(3-(piperidin-1-yl)propyl)-4- tosylpiperazine-1-carboxamide | 408.56 | 408.2 | 409.2 |
| 145. | 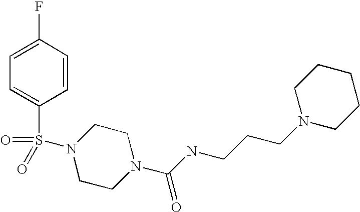 | 4-(4-fluorophenylsulfonyl)-N-(3- (piperidin-1-yl)propyl)piperazine-1- carboxamide | 412.53 | 412.2 | 413.2 |
| 146. | 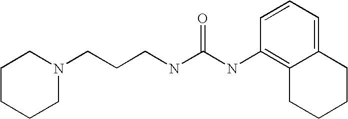 | 1-(3-(piperidin-1-yl)propyl)-3- (5,6,7,8-tetrahydronaphthalen-1- yl)urea | 314.56 | 315.2 | 316.2 |
| 147. | 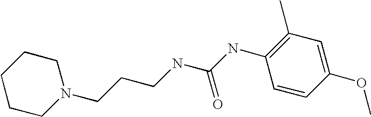 | 1-(4-methoxy-2-methylphenyl)-3-(3- (piperidin-1-yl)propyl)urea | 305.42 | 305.2 | 306.2 |
| 148. | 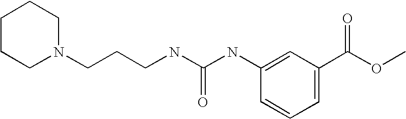 | methyl 3-(3-(3-piperidin-1- yl)propyl)ureido)benzoate | 319.4 | 319.2 | 320.2 |
| 149. | 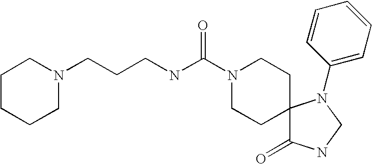 | 4-oxo-1-phenyl-N-(3-(piperidin-1- yl)propyl)-1,3,8- triazaspiro[4.5]decane-8- carboxamide | 399.54 | 399.3 | 400.3 |
| 150. | 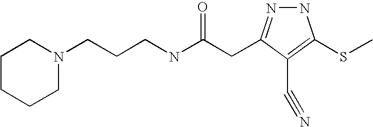 | 1-(4-cyano-5-(methylthio)-1H- pyrazol-3-yl)-3-(3-(piperidin-1- yl)propyl)urea | 322.44 | 322.2 | 323.2 |
| 151. | 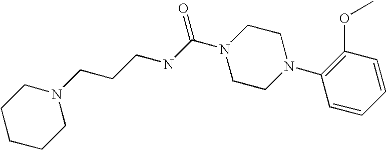 | 4-(2-methoxyphenyl)-N-(3- (piperidin-1-yl)propyl)piperazine-1- carboxamide | 360.5 | 360.3 | 361.3 |
| 152. | 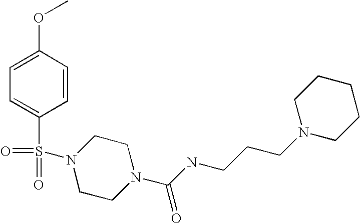 | 4-(4-methoxyphenylsulfonyl)-N-(3- (piperidin-1-yl)propyl)piperazine-1- carboxamide | 424.56 | 424.2 | 425.2 |
| 153. | 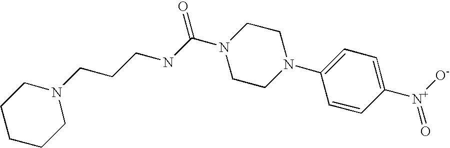 | 4-(4-nitrophenyl)-N-(3-(piperidin-1- yl)propyl)piperazine-1-carboxamide | 375.47 | 375.2 | 376.2 |
| 154. | 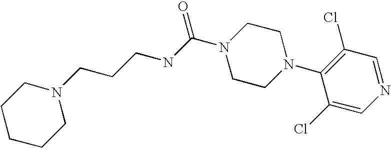 | 4-(3,5-dichloropyridin-4-yl)-N-(3- (piperidin-1-yl)propyl)piperazine-1- carboxamide | 400.35 | 399.2 | 400.2 |
| 155. | 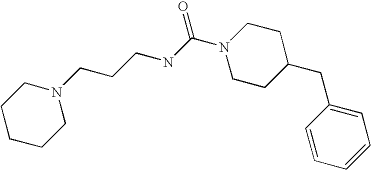 | 4-benzyl-N-(3-(piperidin-1- yl)propyl)piperazine-1-carboxamide | 343.51 | 343.3 | 344.3 |
| 156. | 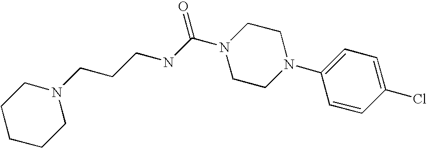 | 4-(4-chlorophenyl)-N-(3-(piperidin- 1-yl)propyl)piperazine-1- carboxamide | 364.92 | 364.2 | 365.2 |
| 157. | 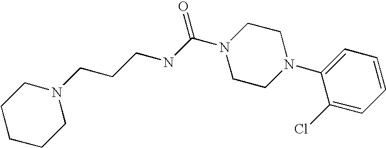 | 4-(2-chlorophenyl)-N-(3-(piperidin- 1-yl)propyl)piperazine-1- carboxamide | 364.92 | 364.2 | 365.2 |
| 158. | 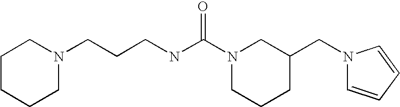 | 3-((1H-pyrrol-1-yl)methyl)-N-(3- (piperidin-1-yl)propyl)piperidine-1- carboxamide | 332.49 | 332.3 | 333.3 |
| 159. | 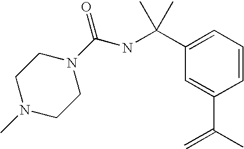 | 4-methyl-N-(2-(3-prop-1-en-2- yl)phenyl)propan-2-yl)piperazine-1- carboxamide | 301.43 | 301.2 | 302.2 |
| 160. | 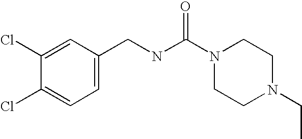 | N-(3,4-dichlorobenzyl)-4- ethylpiperazine-1-carboxamide | 316.23 | 315.1 | 316.1 |
| 161. | 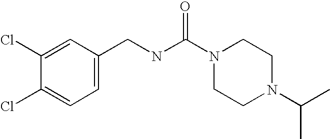 | N-(3,4-dichlorobenzyl)-4- isopropylpiperazine-1-carboxamide | 330.26 | 329.1 | 330.1 |

*High Resolution analytical MS method: Data were acquired in positive ion electrospray mode on an electrospray orthogonal time-of-flight mass spectrometer at a resolution of about 6500. Measurements were made with reversed phase HPLC sample introduction using a linear ACN/water gradient with 0.1% formic acid as the modifier. The experiment was performed using the lockspray accessory with reserpine as the lock mass compound.

In addition, the procedure of Example 1 may be used to prepare all the compounds described earlier in the present specification.

1. A compound of formula I, II or III, a pharmaceutically acceptable salt thereof, diastereomers, enantiomers, or mixtures thereof: 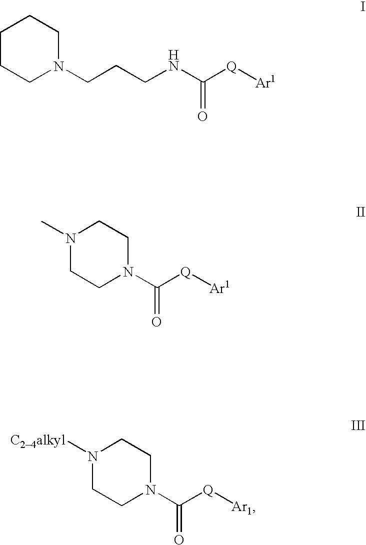
wherein
Ar¹ is selected from C₆₋₁₀aryl and C₂₋₉heteroaryl, wherein said C₆₋₁₀aryl and C₂₋₉heteroaryl are optionally substituted with one or more groups selected from —R, —NO₂, —OR, —Cl, —Br, —I, —F, —CF₃, —OCF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₃₋₆cycloalkyl, C₃₋₆heterocyclyl, phenyl, benzyl, C₁₆alkyl or C₂₋₆alkenyl and wherein said R is further optionally substituted with one or more groups selected from methyl, methoxy, hydroxy and halogen; and
Q is a divalent or trivalent group that connects the carbonyl with Ar¹, wherein said divalent or trivalent group contains at least one nitrogen, said nitrogen is directly connected to the carbonyl group of formula I, II or III to form an amide bond therebetween, and said trivalent group is fused with Ar¹, or Ar¹ is represented by 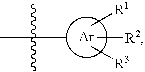
wherein Ar is selected from phenyl, pyridyl, naphthyl, 1,2,3,4-tetrahydro-naphthyl; thienyl, furyl, thiazolyl, benzo[1,3]dioxolyl, 4,5,6,7-tetrahydro-thieno[2,3-c]pyridinyl; 2,3-dihydro-benzo[1,4]dioxinyl; quinolyl; isoquinolyl; indolyl; pyrroyl, benzotriazolyl; benzoimidazolyl, 2,3-dihydro-benzofuranyl; 2,3-dihydro-isoindol-1-on-yl; benzo[1,2,3]thiadiazolyl, benzothiazolyl, and 4H-benzo[1,4]oxazin-3-on-yl;
R¹, R² and R³ are independently selected from —R, —NO₂, —OR, —Cl, —Br, —I, —F, —CF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₅₋₆cycloalkyl, C₃₋₅heterocyclyl, phenyl, benzyl, C₁₋₄alkyl or C₂₋₄alkenyl, and wherein said R is further optionally substituted with one or more groups selected from methyl, methoxy, hydroxy and halogen;
Q is selected from: 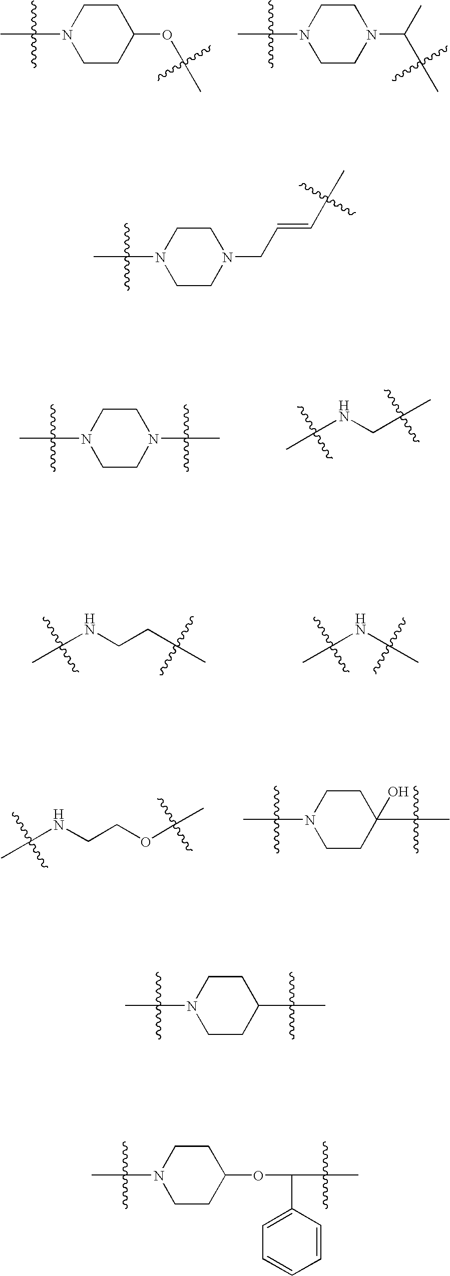 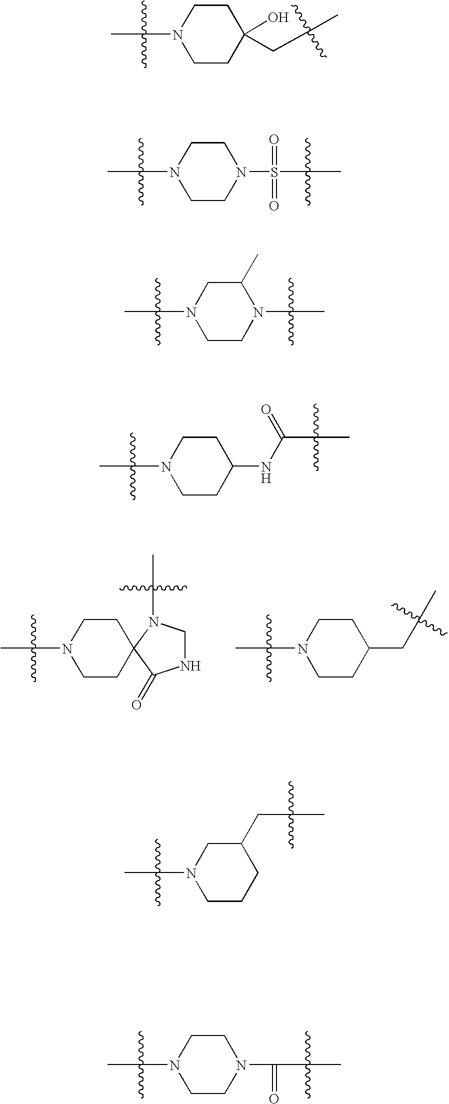
or Q may be a trivalent group such as 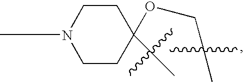
which is fused with Ar¹,
wherein Ar¹ is a divalent aromatic group such as 1,2-phenylene.

2. A compound according to claim 1, wherein Ar¹ is represented by 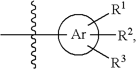
wherein Ar is selected from phenyl, pyridyl, naphthyl, 1,2,3,4-tetrahydro-naphthyl; thienyl, furyl, thiazolyl, benzo[1,3]dioxolyl, 4,5,6,7-tetrahydro-thieno[2,3-c]pyridinyl; 2,3-dihydro-benzo[1,4]dioxinyl; quinolyl; isoquinolyl; indolyl; pyrroyl, benzotriazolyl; benzoimidazolyl, 2,3-dihydro-benzofuranyl; 2,3-dihydro-isoindol-1-on-yl; benzo[1,2,3]thiadiazolyl, benzothiazolyl, and 4H-benzo[1,4]oxazin-3-on-yl;
R¹, R² and R³ are independently selected from —R, —NO₂, —OR, —Cl, —Br, —I, —F, —CF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₅₋₆cycloalkyl, C₃₋₅heterocyclyl, phenyl, benzyl, C₁₋₄alkyl or C₂₋₄alkenyl, and wherein said R is further optionally substituted with one or more groups selected from methyl, methoxy, hydroxy and halogen;
Q is selected from: 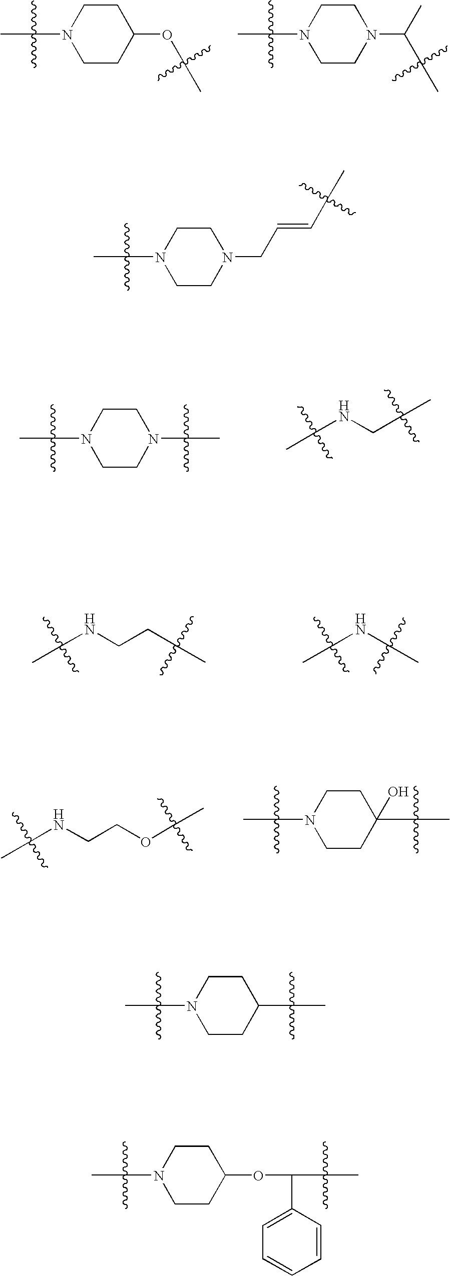 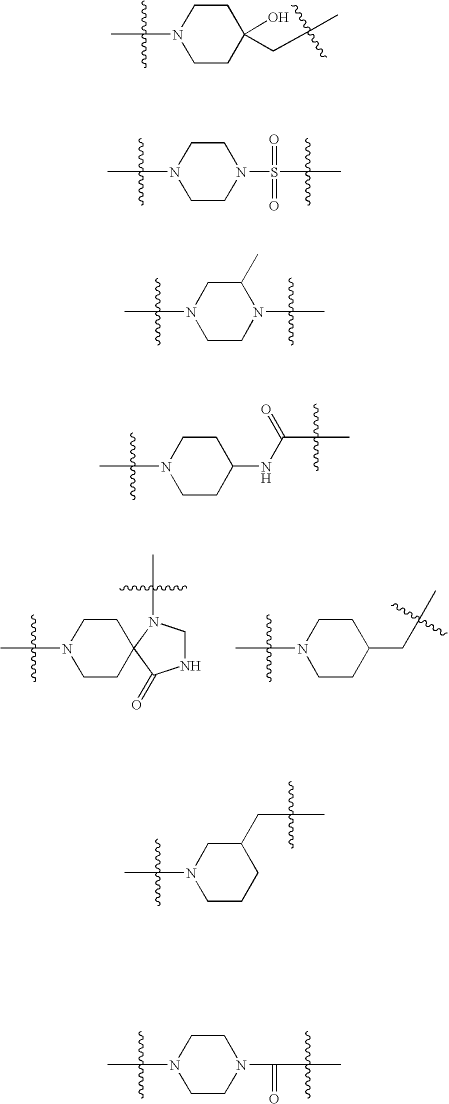
or Q may be a trivalent group such as 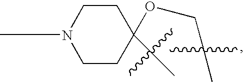
which is fused with Ar¹,
wherein Ar¹ is a divalent aromatic group such as 1,2-phenylene.

3. A compound according to claim 1,
wherein Ar¹ is selected from phenyl, 2-pyridyl, 4-pyridyl; 1-naphthyl, 2-naphthyl, 1,2,3,4-tetrahydro-naphth-1-yl; 1,2,3,4-tetrahydro-naphth-5-yl; 2-thienyl, 3-thienyl, 2-furyl, 2-thiazolyl, benzo[1,3]dioxol-5-yl, 4,5,6,7-tetrahydro-thieno[2,3-c]pyridin-2-yl; 2,3-dihydro-benzo[1,4]dioxin-6-yl; 2,3-dihydro-benzo[1,4]dioxin-2-yl; quinol-2-yl, isoquinol-5-yl; 1H-indol-4-yl, 1H-indol-3-yl, 1H-indol-2-yl, 1H-indol-7-yl, 1-pyrroyl, 1H-benzotriazol-5-yl, 1H-benzoimidazol-5-yl, 2,3-dihydro-benzofuran-5-yl, 2,3-dihydro-isoindol-1-on-2-yl; benzo[1,2,3]thiadiazol-5-yl, benzo[1,2,3]thiadiazol-6-yl, benzothiazol-6-yl, benzothiazol-2-yl, and 4H-benzo[1,4]oxazin-3-on-7-yl, wherein Ar¹ is further optionally substituted with one or more groups selected from C₁₋₄alkyl, C₂₋₄alkenyl, C₁₋₄alkoxy, C₁₋₄alkenyloxy, phenoxy, 4-methoxyphenoxy, benzyl, acetoamino, methylsulfonyl, methoxycarbonyl, nitro, chloro, fluoro, bromo, iodo, 1-pyrroyl, 2-methyl-pyrro-1-yl, amino, phenylsulfonyl, aceto, 1-piperidinyl, [1,2,3]thiadiazol-4-yl, 4-morpholinyl, methoxy, ethoxy, isopropyloxy, methythio, cyano, dimethylamino, hydroxy, methylaminosulfonyl, trifluoromethyl, trifluoromethoxy, phenyl, 4-hydroxyl-phenyl, diethylamino, methylsulfonyl, aminosulfonyl, cyclohexyl, 1-pyrrolyl, 1H-pyrazol-3-yl, 5-tetrazolyl, 1-piperidinyl, 1-pyrazolyl, methylsulfonylmethyl, 3,5-dimethyl-pyrazolyl, pyrrolidin-2-on-1-yl; and
Q is selected from

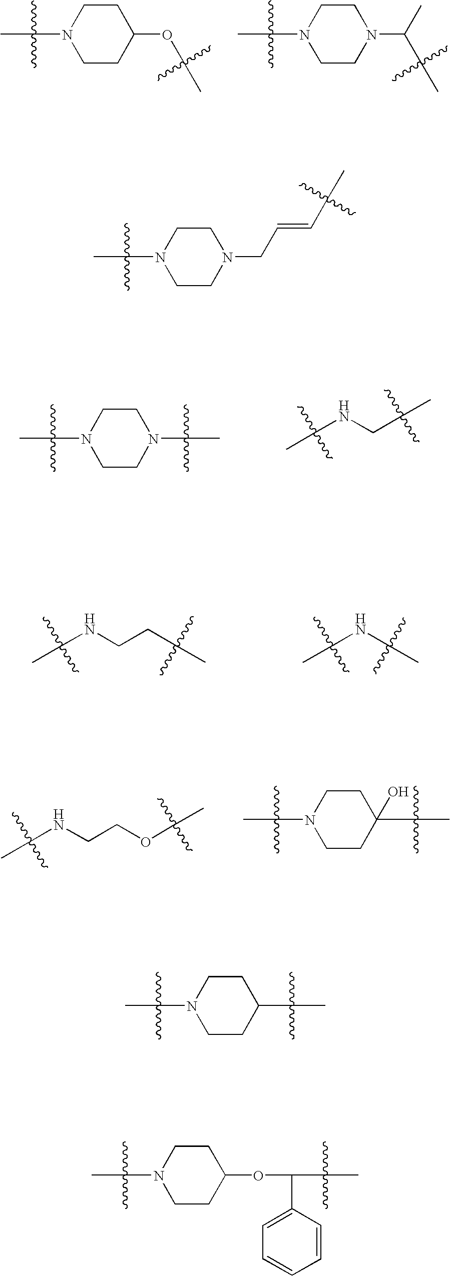

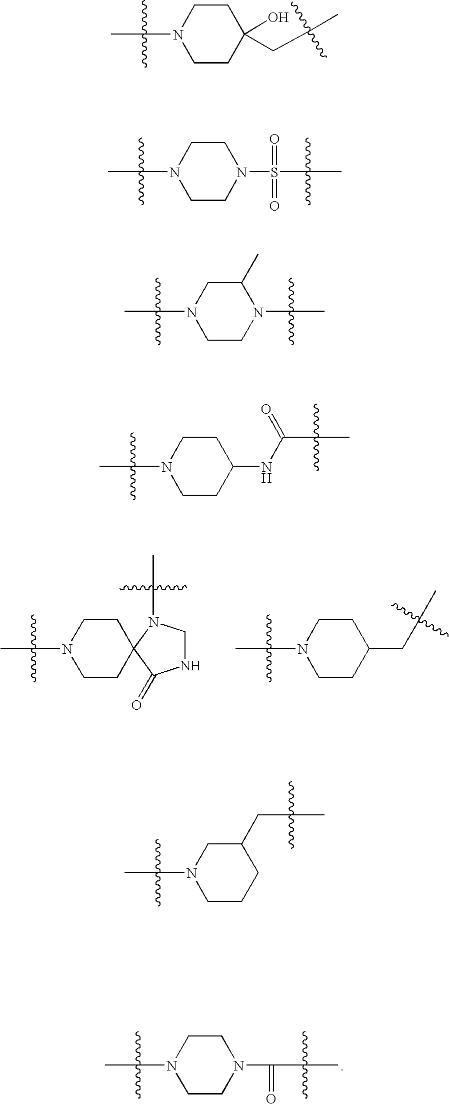

4. A compound selected from: 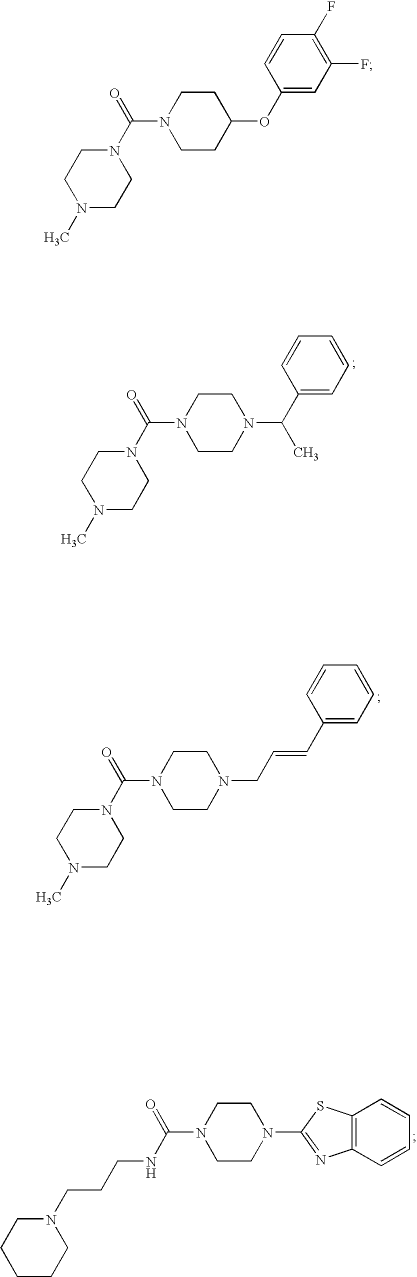 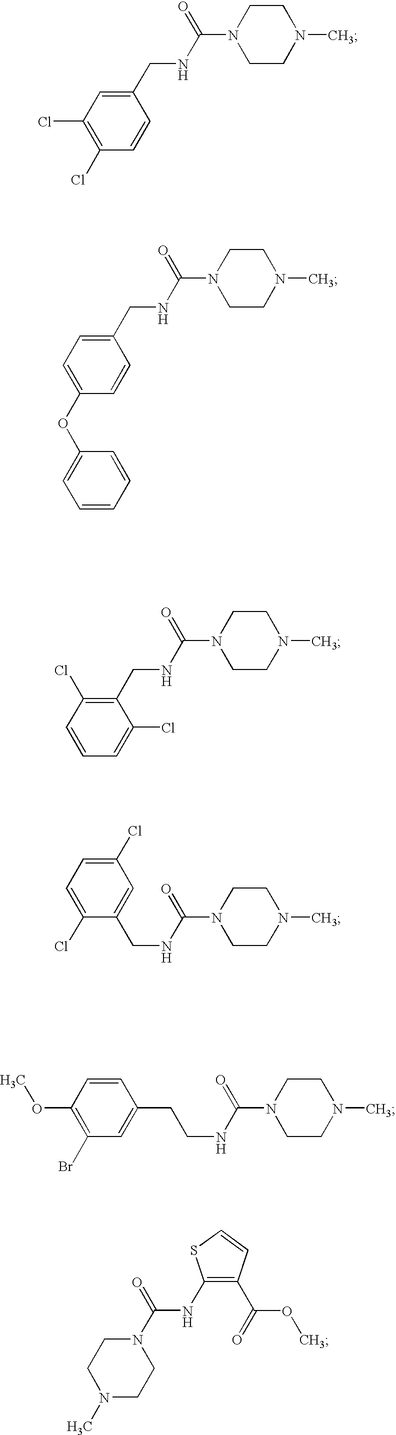 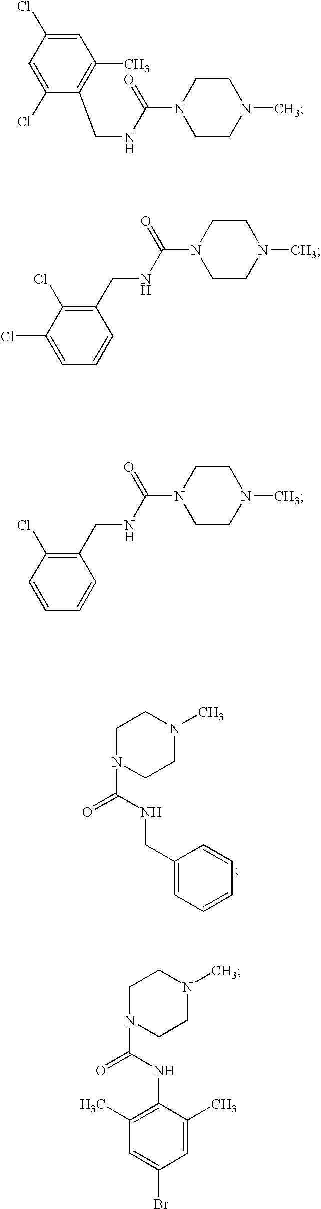 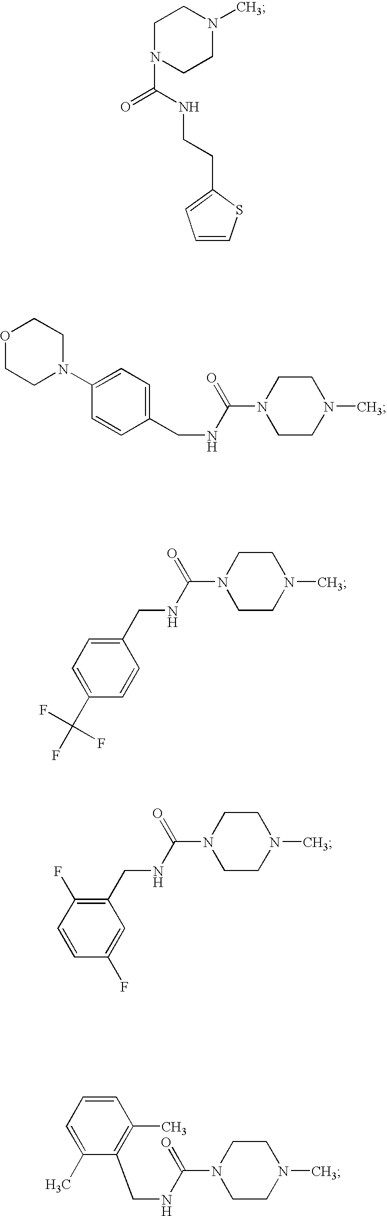 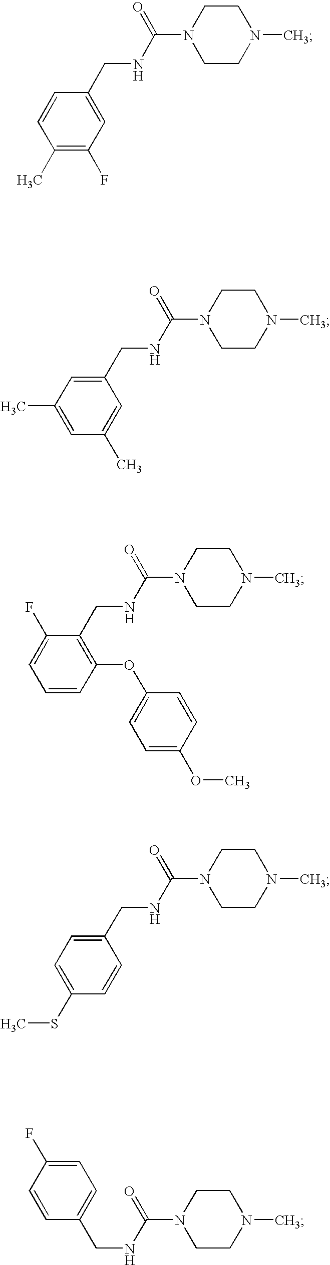 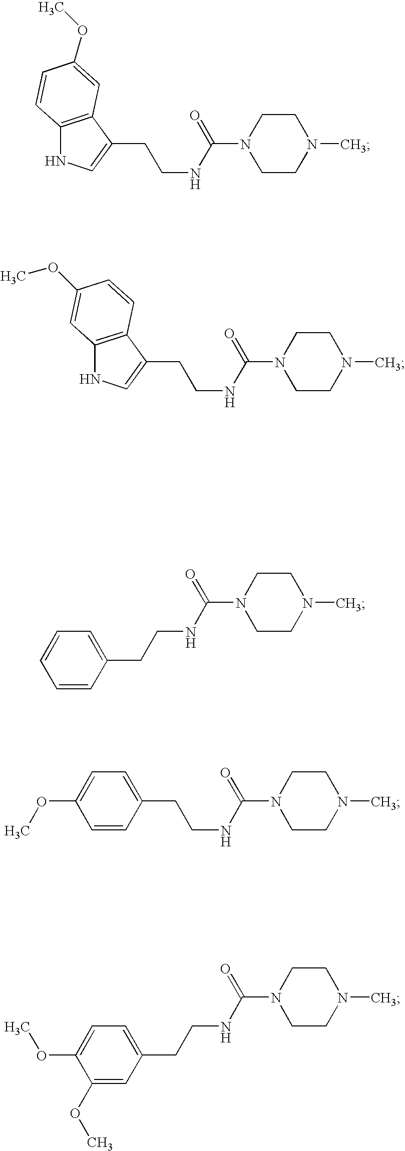 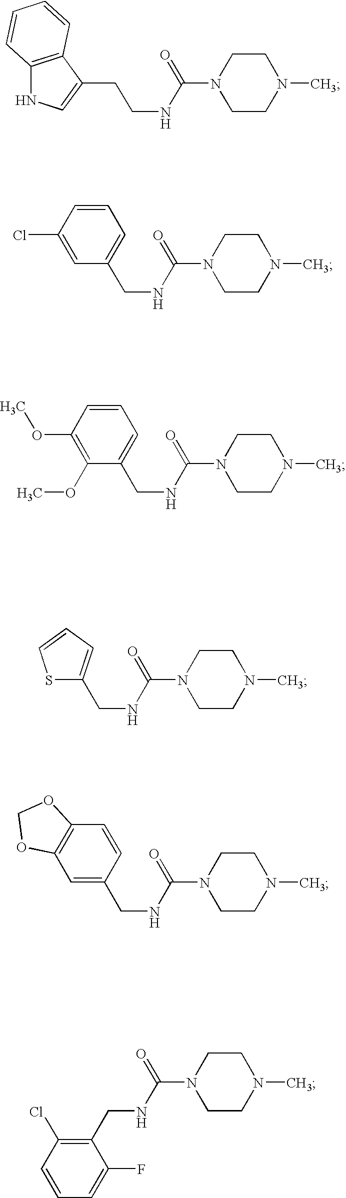 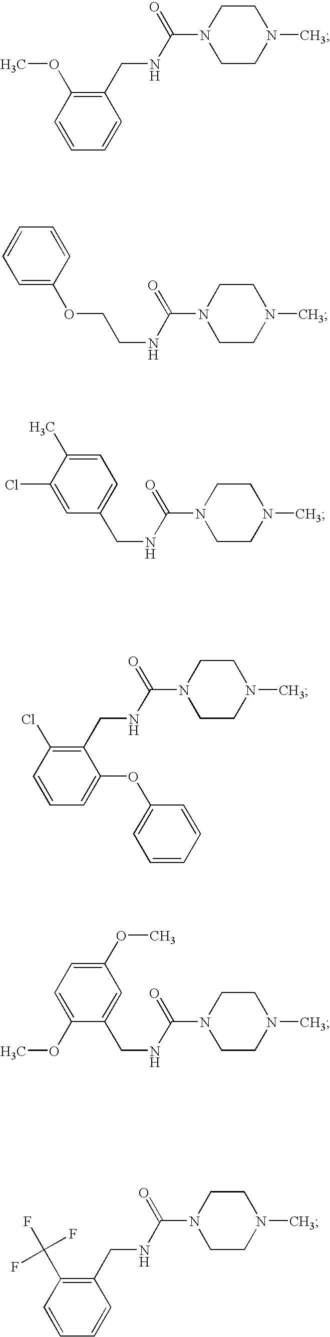 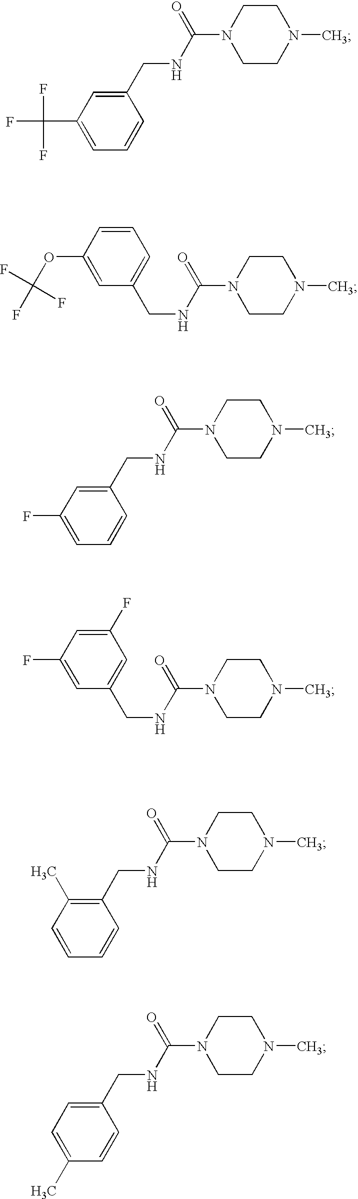 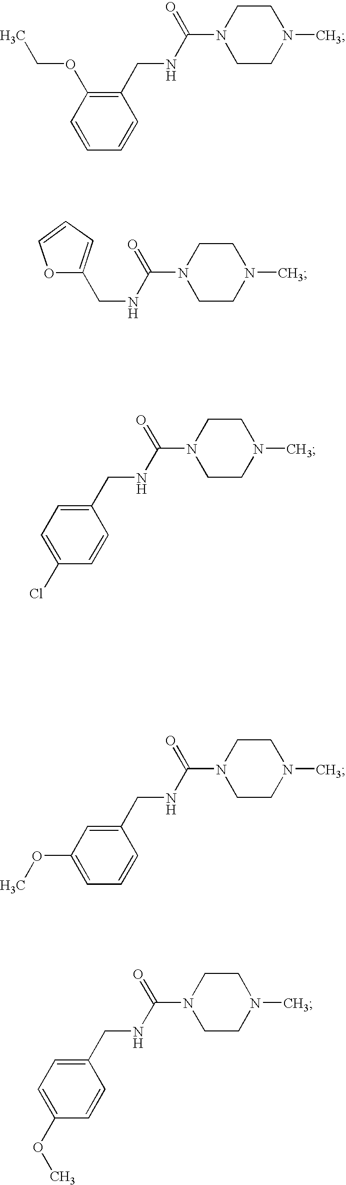 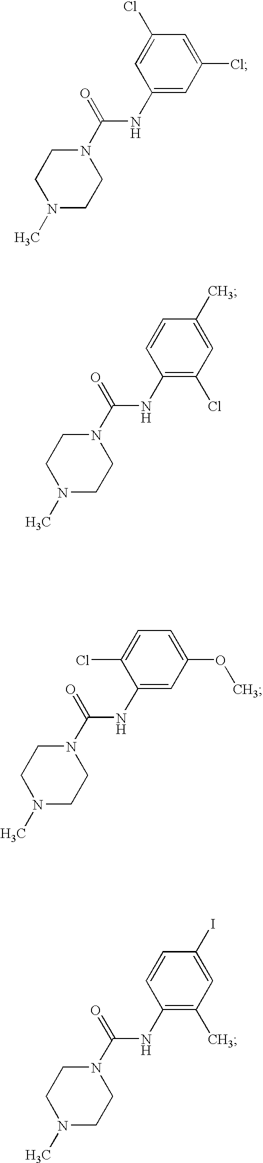 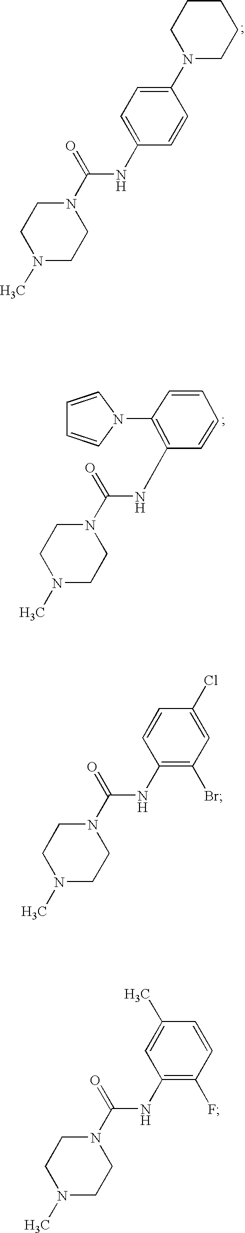 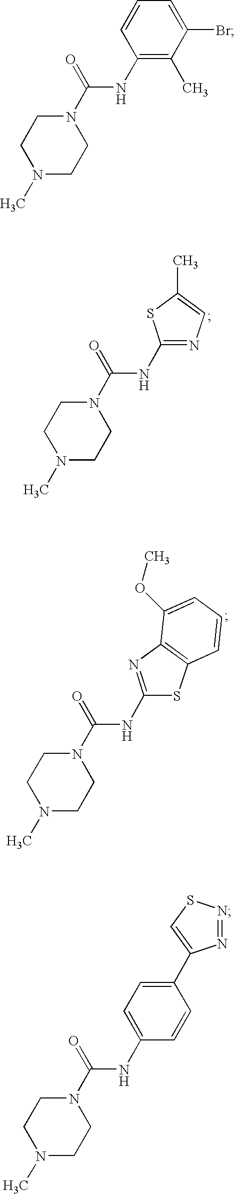 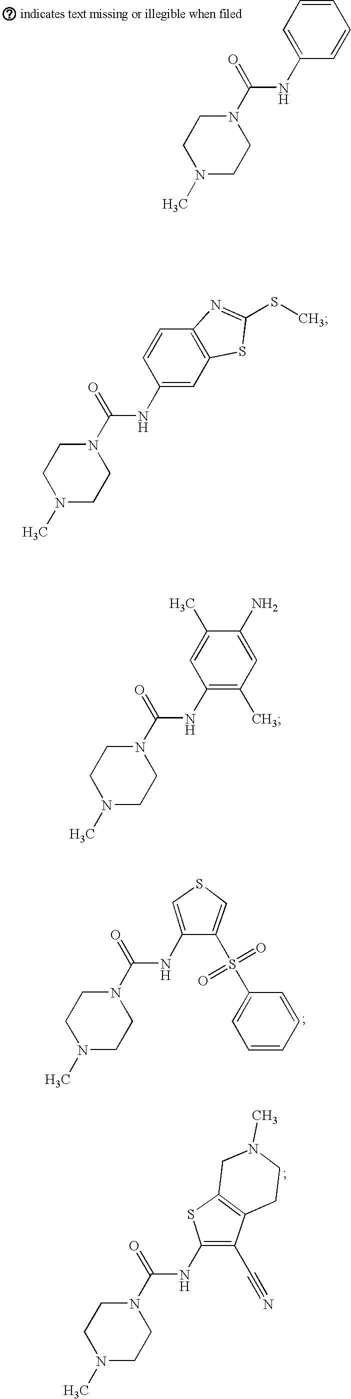 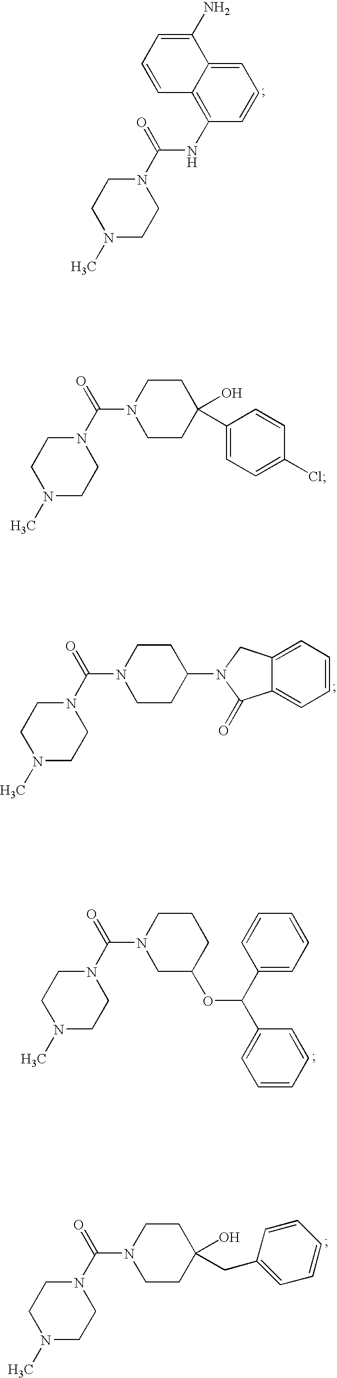 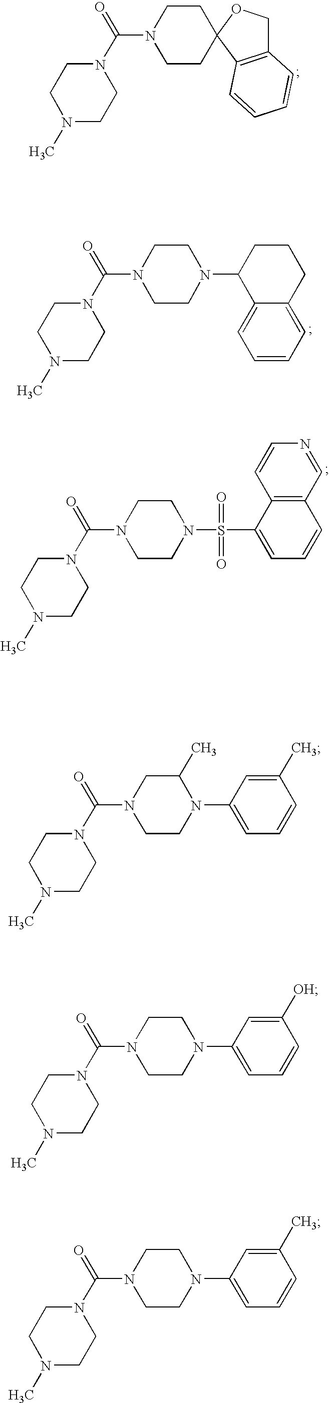 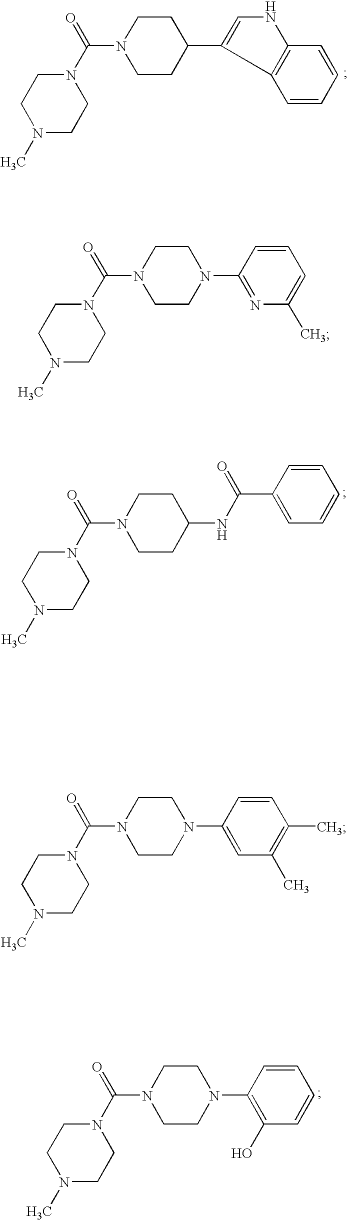 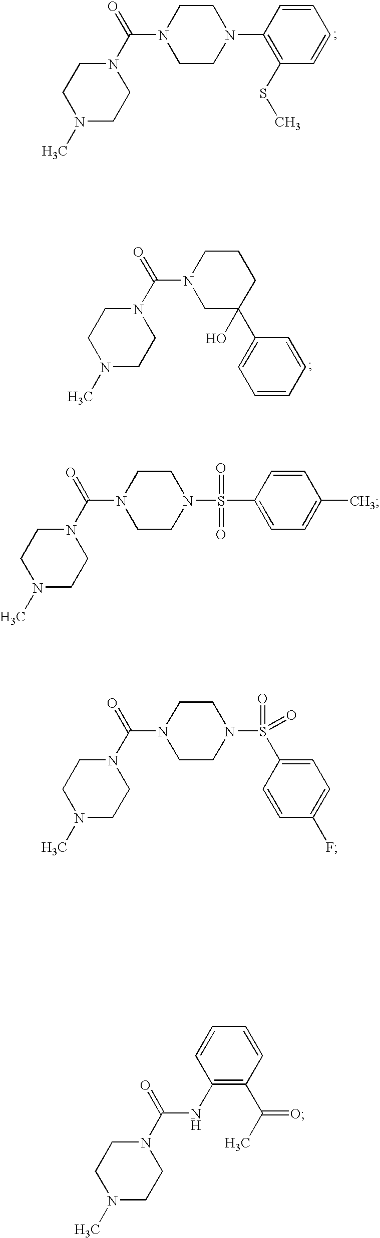 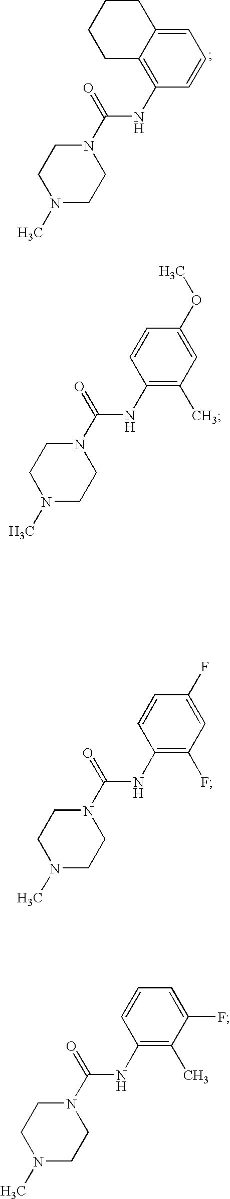 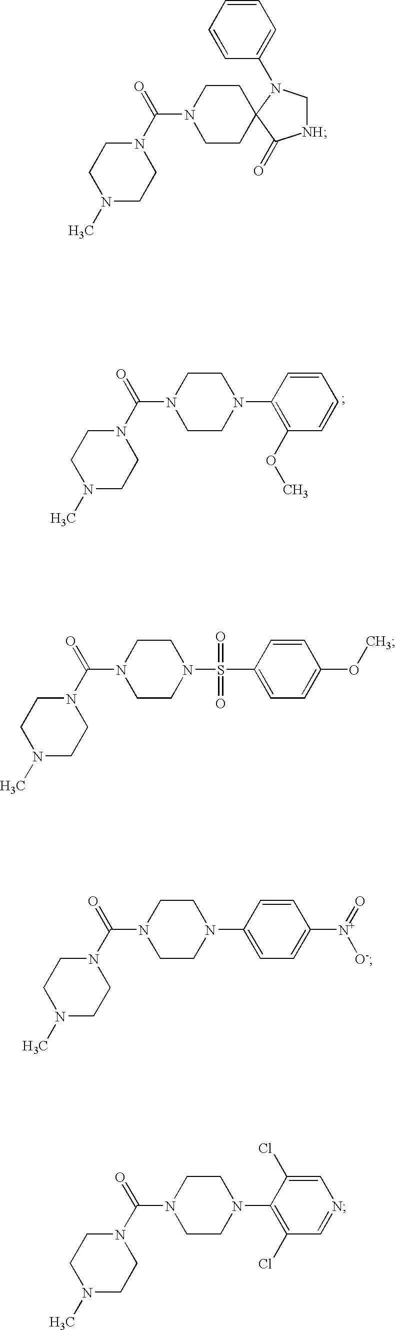 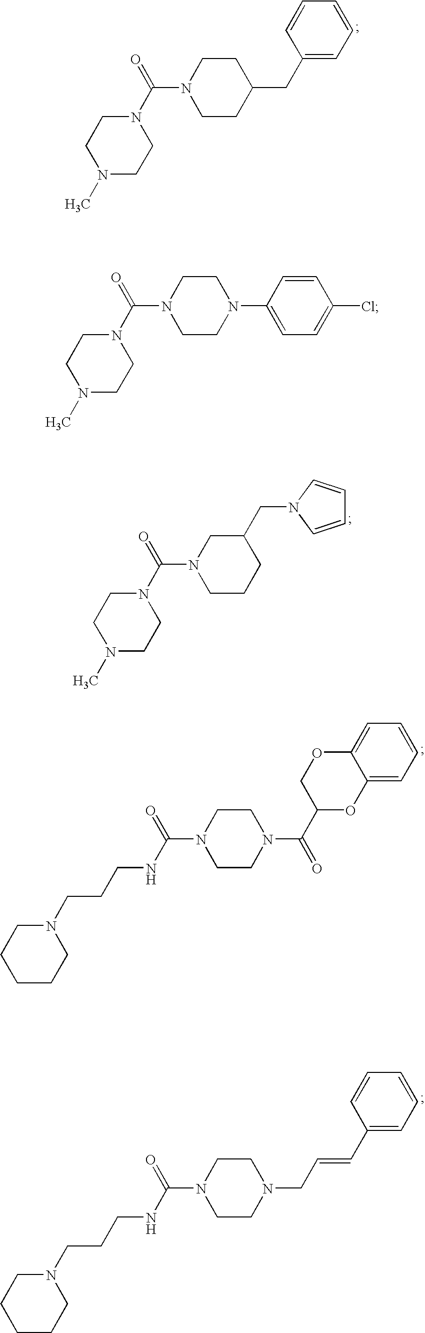 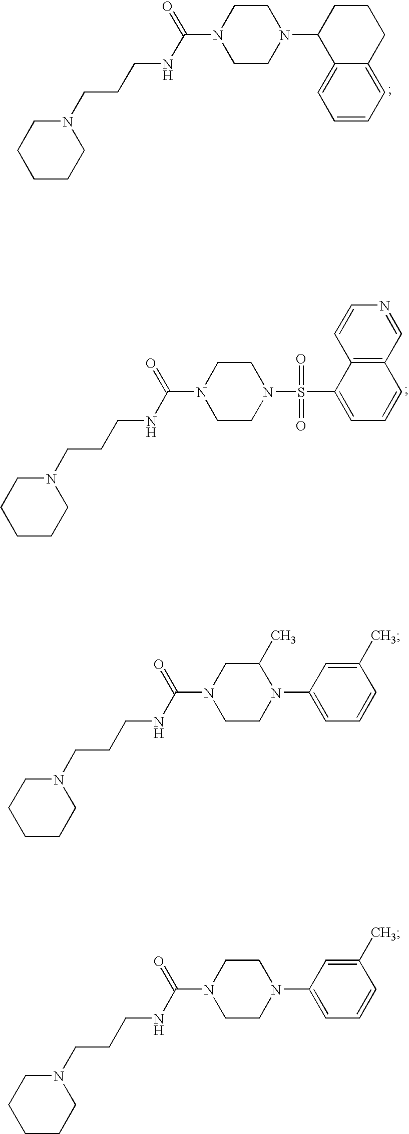 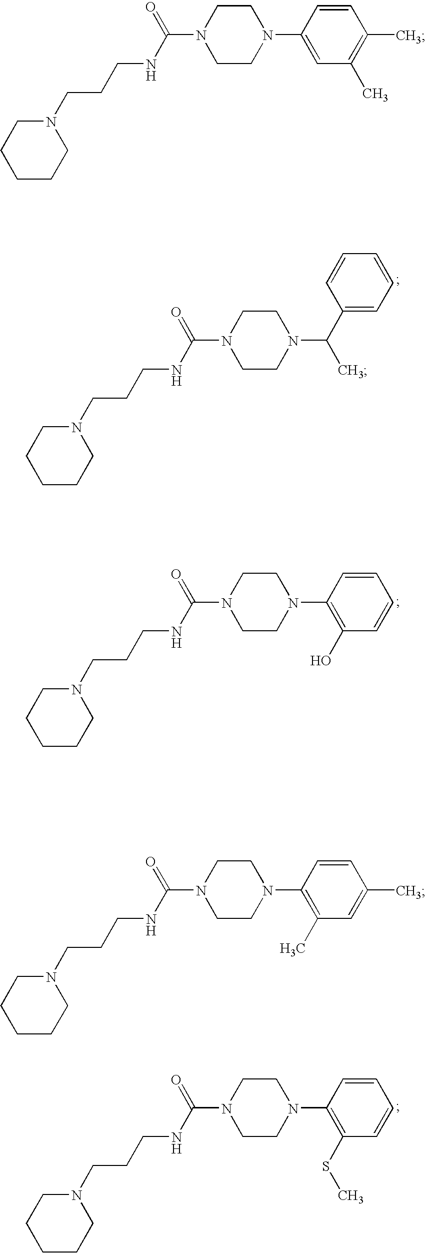 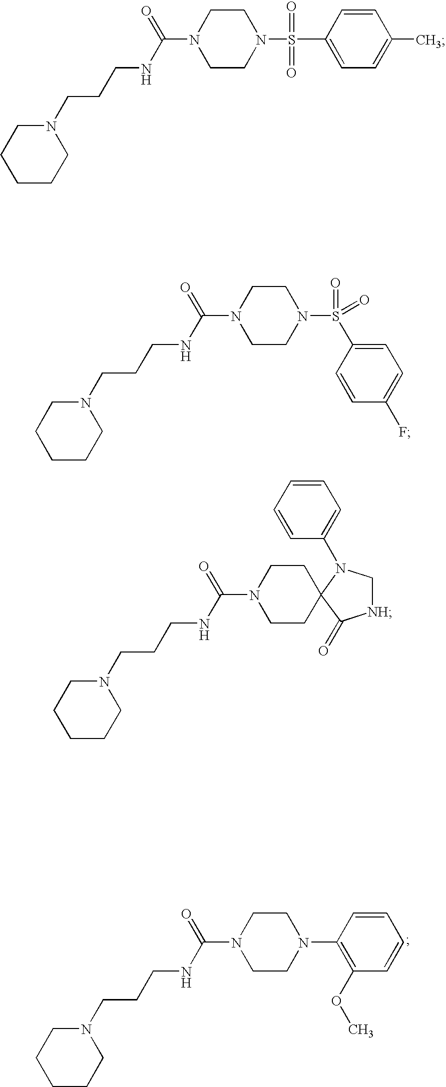 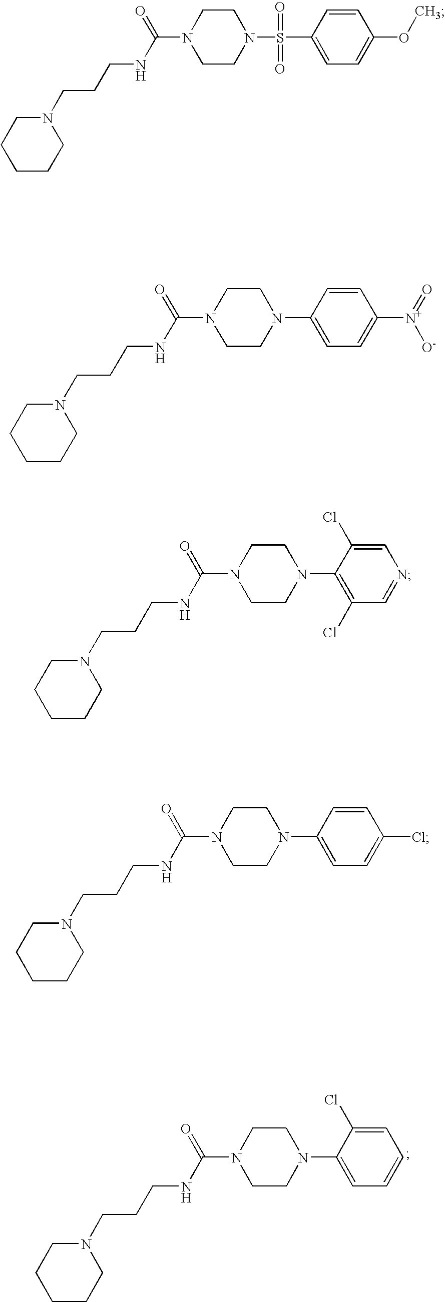
and pharmaceutically acceptable salts thereof.

5-6. (canceled)

7. A pharmaceutical composition comprising a compound according to claim 1 and a pharmaceutically acceptable carrier.

8. A method for the therapy of depression in a warm-blooded animal, comprising the step of administering to said animal in need of such therapy a therapeutically effective amount of a compound according to claim 1.

9. A process for preparing a compound of formula I, comprising: 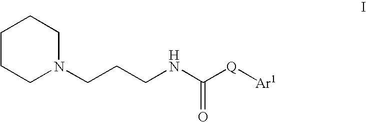
reacting Ar¹-Q-H with 3-(1-piperidino)propylamine and a haloformate,
Ar¹ is selected from C₆₋₁₀aryl and C₂₋₉heteroaryl, wherein said C₆₋₁₀aryl and C₂₋₉heteroaryl are optionally substituted with one or more groups selected from —R, —NO₂, —OR, —Cl, —Br, —I, —F, —CF₃, —OCF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₃₋₆cycloalkyl, C₃₋₆heterocyclyl, phenyl, benzyl, C₁₋₆alkyl or C₂₋₆alkenyl and wherein said R is further optionally substituted with one or more groups selected from methyl, methoxy hydroxy and halogen;
Q is a divalent or trivalent group that connects the carbonyl with Ar¹, wherein said divalent or trivalent group contains at least one nitrogen wherein said nitrogen of Q is connected to the H in Ar¹-Q-H to form an amino, and said trivalent group is fused with Ar¹; and said Q-H of Ar¹-Q-H forms an amino group.

10. A process for preparing a compound of formula I, comprising: 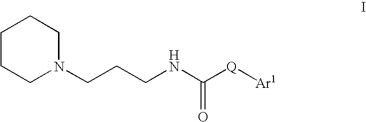
combining Ar¹-Q-H with 3-(1-piperidino)propylamine and a haloformate,
wherein Ar¹ is selected from phenyl, 2-pyridyl, 4-pyridyl; 1-naphthyl, 2-naphthyl, 1,2,3,4-tetrahydro-naphth-1-yl; 1,2,3,4-tetrahydro-naphth-5-yl; 2-thienyl, 3-thienyl, 2-furyl, 2-thiazolyl, benzo[1,3]dioxol-5-yl, 4,5,6,7-tetrahydro-thieno[2,3-c]pyridin-2-yl; 2,3-dihydro-benzo[1,4]dioxin-6-yl; 2,3-dihydro-benzo[1,4]dioxin-2-yl; quinol-2-yl, isoquinol-5-yl; 1H-indol-4-yl, 1H-indol-3-yl, 1H-indol-2-yl, 1H-indol-7-yl, 1-pyrroyl, 1H-benzotriazol-5-yl, 1H-benzoimidazol-5-yl, 2,3-dihydro-benzofuran-5-yl, 2,3-dihydro-isoindol-1-on-2-yl; benzo[1,2,3]thiadiazol-5-yl, benzo[1,2,3]thiadiazol-6-yl, benzothiazol-6-yl, benzothiazol-2-yl, and 4H-benzo[1,4]oxazin-3-on-7-yl, wherein Ar¹ is further optionally substituted with one or more groups selected from C₁₋₄alkyl, C₂₋₄alkenyl, C₁₋₄alkoxy, C₁₋₄alkenyloxy, phenoxy, 4-methoxyphenoxy, benzyl, acetoamino, methylsulfonyl, methoxycarbonyl, nitro, chloro, fluoro, bromo, iodo, 1-pyrroyl, 2-methyl-pyrro-1-yl, amino, phenylsulfonyl, aceto, 1-piperidinyl, [1,2,3]thiadiazol-4-yl, 4-morpholinyl, methoxy, ethoxy, isopropyloxy, methythio, cyano, dimethylamino, hydroxy, methylaminosulfonyl, trifluoromethyl, trifluoromethoxy, phenyl, 4-hydroxyl-phenyl, diethylamino, aminosulfonyl, cyclohexyl, 1-pyrrolyl, 1H-pyrazol-3-yl, 5-tetrazolyl, 1-piperidinyl, 1-pyrazolyl, methylsulfonylmethyl, 3,5-dimethyl-pyrazolyl, pyrrolidin-2-on-1-yl; and
Q is selected from 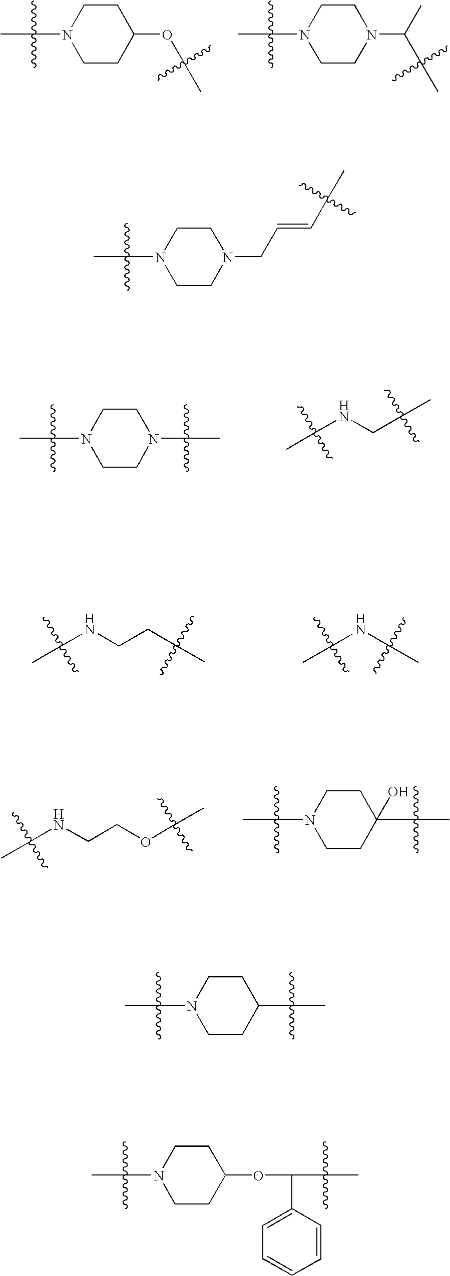 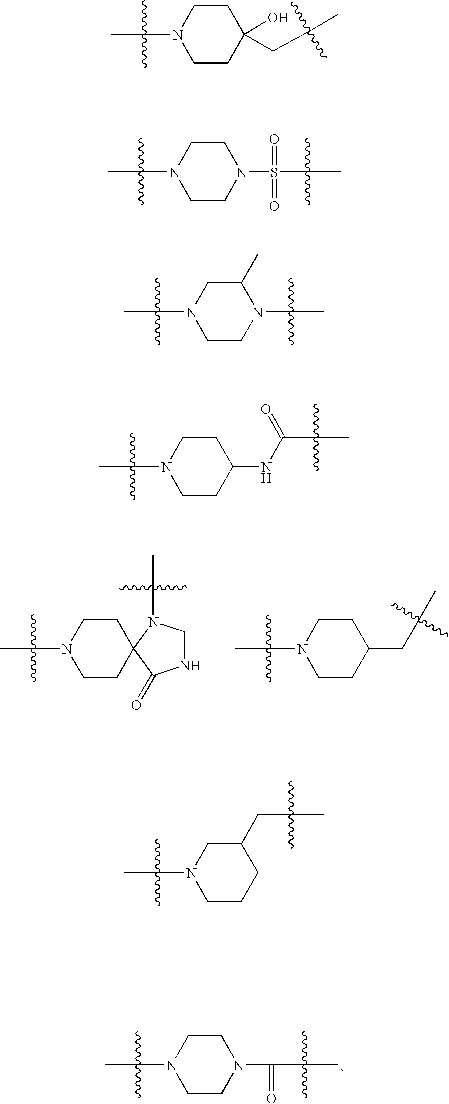
and wherein the left side nitrogen atom in the above-identified structures of Q is connected to the H in Ar¹-Q-H to form an amino group.

11. A process for preparing a compound of formula II, comprising: 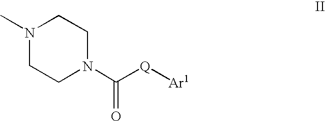
reacting Ar¹-Q-H with N-methyl piperazine and a haloformate,
Ar¹ is selected from C₆₋₁₀aryl and C₂₋₉heteroaryl, wherein said C₆₋₁₀aryl and C₂₋₉heteroaryl are optionally substituted with one or more groups selected from —R, —NO₂, —OR, —Cl, —Br, —I, —F, —CF₃, —OCF₃, —C(═O)R, —C(═O)OH, —NH₂, —SH, —NHR, —NR₂, —SR, —SO₃H, —SO₂R, —SO₂NR, —S(═O)R, —CN, —OH, —C(═O)OR, —C(═O)NR₂, —NRC(═O)R, and —NRC(═O)—OR, wherein R is, independently, a hydrogen, C₃₋₆cycloalkyl, C₃₋₆heterocyclyl, phenyl, benzyl, C₁₋₆alkyl or C₂₋₆alkenyl and wherein said R is further optionally substituted with one or more groups selected from methyl, methoxy hydroxy and halogen;
Q is a divalent or trivalent group that connects the carbonyl with Ar¹, wherein said divalent or trivalent group contains at least one nitrogen, wherein said nitrogen of Q is connected to the H in Ar¹-Q-H to form an amino, and said trivalent group is fused with Ar¹; and said Q-H of Ar¹-Q-H forms an amino group.

12. A process of preparing a compound of formula II, comprising: 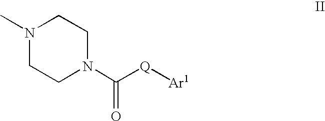
combining Ar¹-Q-H with N-methyl piperazine and a haloformate,
wherein Ar¹ is selected from phenyl, 2-pyridyl, 4-pyridyl; 1-naphthyl, 2-naphthyl, 1,2,3,4-tetrahydro-naphth-1-yl; 1,2,3,4-tetrahydro-naphth-5-yl; 2-thienyl, 3-thienyl, 2-furyl, 2-thiazolyl, benzo[1,3]dioxol-5-yl, 4,5,6,7-tetrahydro-thieno[2,3-c]pyridin-2-yl; 2,3-dihydro-benzo[1,4]dioxin-6-yl; 2,3-dihydro-benzo[1,4]dioxin-2-yl; quinol-2-yl, isoquinol-5-yl; 1H-indol-4-yl, 1H-indol-3-yl, 1H-indol-2-yl, 1H-indol-7-yl, 1-pyrroyl, 1H-benzotriazol-5-yl, 1H-benzoimidazol-5-yl, 2,3-dihydro-benzofuran-5-yl, 2,3-dihydro-isoindol-1-on-2-yl; benzo[1,2,3]thiadiazol-5-yl, benzo[1,2,3]thiadiazol-6-yl, benzothiazol-6-yl, benzothiazol-2-yl, and 4H-benzo[1,4]oxazin-3-on-7-yl, wherein Ar¹ is further optionally substituted with one or more groups selected from C₁₋₄alkyl, C₂₋₄alkenyl, C₁₋₄alkoxy, C₁₋₄alkenyloxy, phenoxy, 4-methoxyphenoxy, benzyl, acetoamino, methylsulfonyl, methoxycarbonyl, nitro, chloro, fluoro, bromo, iodo, 1-pyrroyl, 2-methyl-pyrro-1-yl, amino, phenylsulfonyl, aceto, 1-piperidinyl, [1,2,3]thiadiazol-4-yl, 4-morpholinyl, methoxy, ethoxy, isopropyloxy, methythio, cyano, dimethylamino, hydroxy, methylaminosulfonyl, trifluoromethyl, trifluoromethoxy, phenyl, 4-hydroxyl-phenyl, diethylamino, aminosulfonyl, cyclohexyl, 1-pyrrolyl, 1H-pyrazol-3-yl, 5-tetrazolyl, 1-piperidinyl, 1-pyrazolyl, methylsulfonylmethyl, 3,5-dimethyl-pyrazolyl, pyrrolidin-2-on-1-yl; and
Q is selected from 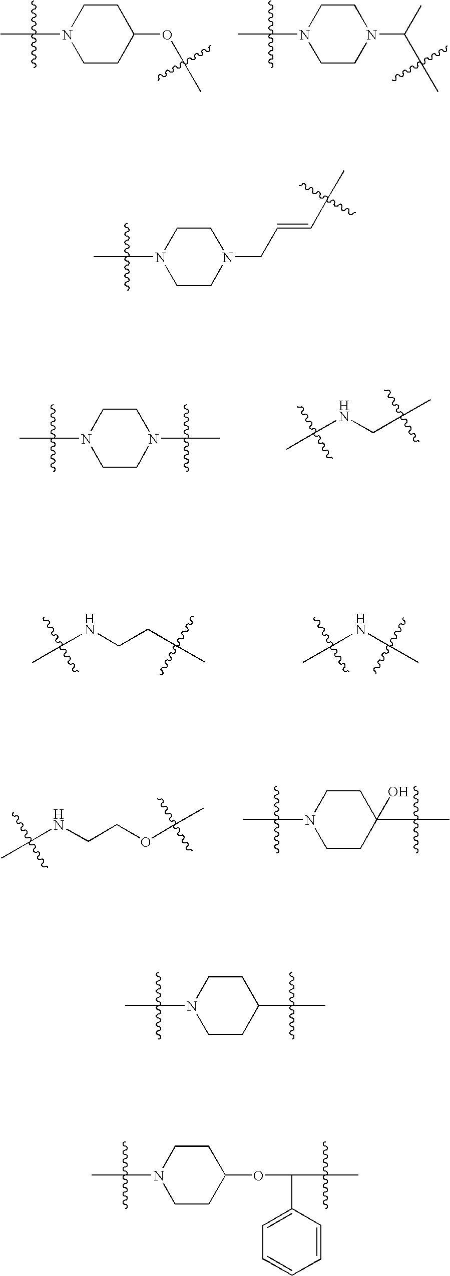 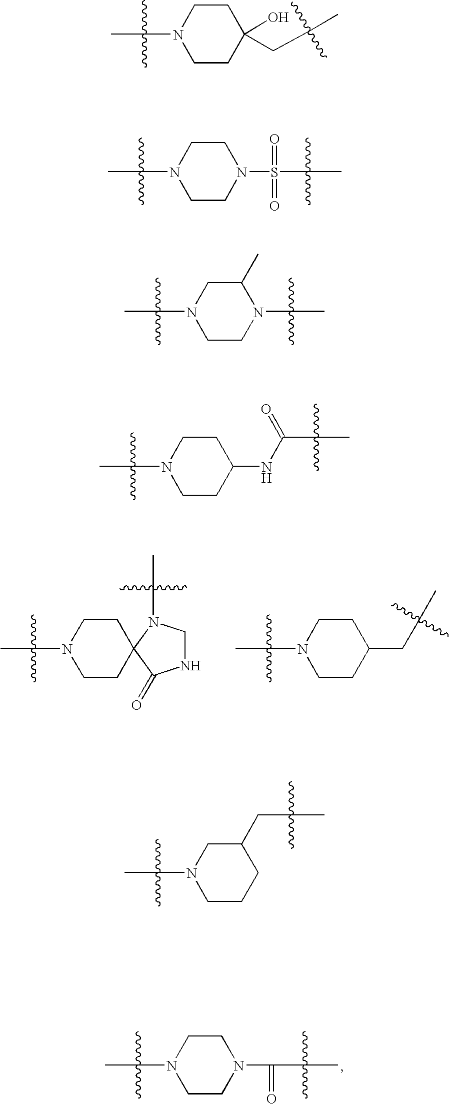
and wherein the left side nitrogen atom in the above-identified structures of Q is connected to the H in Ar¹-Q-H to form an amino group.
